(19) United States
(12) Patent Application Publication
(10) Pub. No.: US 2022/0313873 A1
(43) Pub. Date: Oct. 6, 2022

(54) COLORED BIOLOGIC WOUND TREATMENT PROVIDING HEALING PROGRESS MONITORING

(71) Applicant: KERECIS HF, Isafjordur (IS)

(72) Inventors: Gudmundur Fertram SIGURJONSSON, Arlington, VA (US); Gunnar JOHANNSSON, Isafjordur (IS)

(21) Appl. No.: 17/703,650

(22) Filed: Mar. 24, 2022

Related U.S. Application Data
(60) Provisional application No. 63/165,630, filed on Mar. 24, 2021.

(51) Int. Cl. A61L 27/60; A61L 27/36; C09B 21/00; C09B 11/10

(52) U.S. Cl. CPC A61L 27/60; A61L 27/362; A61L 27/3633; C09B 21/00; C09B 11/10; A61L 2300/442

(57) ABSTRACT

Tissue-regenerating wound treatments, methods of producing tissue-regenerating wound treatments, and methods of treating a wound using a tissue-regenerating wound treatment are provided. The tissue-regenerating wound treatment includes a skin substitute and a coloring agent added to the skin substitute. The coloring agent is a biocompatible coloring agent that degrades upon protease attack within a treated wound.

### CROSS-REFERENCE TO RELATED APPLICATIONS

This application claims the benefit of priority from U.S. provisional application No. 63/165,630, filed on Mar. 24, 2021, the entirety of which is incorporated herein by reference.

### FIELD OF THE DISCLOSURE

The disclosure relates generally to wound treatments and methods for stabilizing, protecting, and/or healing damaged tissue, and particularly to wound treatments and methods that indicate whether ingrowth into a skin substitute of the wound treatment has occurred.

### BACKGROUND

Healthy skin serves several distinct functions, including protecting underlying tissues from abrasion, microbes, water loss, and ultraviolet light damage. The nervous system of healthy, normal skin also provides tactile sensations of touch, pressure, and vibration, thermal sensations of heat and cold, and pain sensations. A body's thermoregulation relies on the skin's ability to sweat and control blood flow to the skin to increase or decrease heat loss. Healthy skin includes three distinct tissue layers: a thin outer layer of cells called the epidermis, a thicker middle layer of connective tissue called the dermis, and an inner, subcutaneous layer. The thin outer layers of the epidermis are composed of flattened, cornified, dead keratinocytes that form a barrier to water loss and microbe entry. The dead keratinocytes are derived from live keratinocytes in the basal layer, which lies above the dermis, and are responsible for skin reepithelization. The epidermis does not contain nerves or blood vessels and obtains water and nutrients through diffusion from the dermis. The dermis, which lies below the epidermis, is composed mostly of collagen fibers and some elastic fibers both produced by fibroblasts and, along with water and large proteoglycan molecules, makes up the extracellular matrix (ECM). This skin layer provides mechanical strength and a substrate for water and nutrient diffusion. It contains blood vessels, nerves, sweat glands, hair follicles, and cells involved in immune function, growth, and repair. The subcutaneous layer is composed of adipocytes that form a thick layer of adipose tissue.

A wound may be considered a disruption of the skin's structural and functional integrity. Thus, a “wound” may include those injuries that cause, for example, cutting, tearing, and/or breaking of the skin such as lacerations, abrasions, incisions, punctures, avulsions, burns, or other such injuries.

After hemostasis, which often follows a wound event, a wound goes through main stages when healing: inflammation, proliferation and remodeling. Chronic wounds may be considered wounds that have failed to pass through the normal healing process in an orderly and timely manner. Chronic wounds often remain in the inflammation phase.

Often, in the cases of significant wounds, such as wounds extending over large areas or in deep wounds, or large or severe burn wounds, or in the case of chronic wounds, skin substitutes are often used to aid in the healing process of the wound and to more quickly restore at least some of the above-noted functions of healthy skin. Skin substitutes may be considered broadly as a group of elements or materials that enable the temporary or permanent occlusion of a wound. Skin substitutes can generally be divided into biological skin substitutes, synthetic skin substitutes, or a hybrid skin substitute that includes biological and synthetic skin substitutes.

Biological skin substitutes often have a more intact extracellular matrix structure, while the synthetic skin substitutes can be synthesized on demand and can be modulated for specific purposes. Biological skin substitutes and synthetic skin substitutes each have advantages and disadvantages. The biological skin substitutes may allow the construction of a more natural new dermis and allow excellent re-epithelialization characteristics due to the presence of a basement membrane. Synthetic skin substitutes may be chemically synthesized and provide the advantages of increase control over scaffold composition. Synthetic skin substitutes include synthetic biolayers including, for example, a synthesized collagen or protein-based matrix or a collagen or protein-based components combined with silicone components. Hybrid skin substitutes may be partly synthesized or produced by living cells and partly chemically synthesized.

Whether biological, synthetic, or hybrid skin substitutes are used, the object of using skin substitutes is to provide an effective, timely, and scar-free wound healing with as much return to the functions of the skin before the wound event.

Examples of commercially available synthetic skin substitutes include Biobrane®, Dermagraft®, Integra®, Apligraf®, MatriDerm®, OrCel®, Hyalomatrix®, and Renoskin®.

U.S. Published Patent Application No. 2003/0059460 discloses a hybrid polymer skin substitute material comprising synthetic and natural polymers that can be used in regenerating living body tissue. The hybrid comprises a cross-linked naturally-occurring polymer and a biodegradation-absorbable synthetic polymer. A series of complicated process steps, however, must be undertaken to produce the hybrid material. In addition, the resulting hybrid material contains synthetic as well as naturally-occurring materials.

Most modern wound treatment products are so called wet-to-dry wound dressings that facilitate improved wound healing by keeping an appropriate moisture level on the wound. The products typically accumulate wound exudate and are exchanged at regular interval.

Biological skin substitutes may include, but are not limited to, skin grafts, including autologous skin grafts, syngeneic skin grafts, allogeneic skin grafts, xenogeneic skin grafts such as porcine skin grafts, cadaveric skin allografts, and amniotic tissue grafts.

Further, in recent years a new class of biological skin graft products has emerged that is intended to improve the micro milieu of the wound by providing the proliferating cells with shelter. Typically the new products are made from biologic materials containing intact collagen or reconstituted collagens. Examples include brands such as; Oasis, Matristem, Integra and Puracol. Those products are often referred to by clinicians as being matrix products. The matrix products are inserted into the wound where they are to attract cellular ingrowth. A secondary wet-to-dry wound dressing is then applied on top of the wound dressing. One example of a matrix product derived from intact, decellularized fish skin is described in U.S. Pat. No. 8,613,957 B2, granted Dec. 24, 2013, and incorporated herein by reference in its entirety. The decellularized fish skin product describes by U.S. Pat. No. 8,613,957 serves as a scaffold material that provides an intact scaffold for support for ingrowth of endothelial and/or epithelial cells. The decellularized fish skin scaffold material is biocompatible thus can be integrated by the host. Omega3 Wound is a commercially available skin substitute made from the minimally processed skin of wildcaught Atlantic cod originating from Iceland. The fish skin is structurally alike to human skin with three basic layers including epidermis, dermis, and hypodermis and contains proteins, lipids, fatty acids, and other bioactive compounds that are homologous to human skin.

Examples of other biological skin substitutes include those described in U.S. Pat. No. 6,541,023, which describes the use of porous collagen gels derived from fish skin for use as tissue engineering scaffolds. Preparation of the collagen gels involves grinding the fish skin. Additionally, Chinese Patent No. 1068703 describes a process for preparing fish skin for dressing burn wounds, involving separating fish skin from the fish body and placing the skin in a preservation solution of iodine tincture, ethanol, borneol, sulfadiazine zinc and hydrochloric acid in amounts sufficient to establish a pH value of 2.5-3. However, these products can be difficult to handle as the product of U.S. Pat. No. 6,541,023 is in a gel form and the product of China Patent No. 1068703 is stored in a solution.

In addition, a number of extracellular matrix products for medical uses have been derived from human skin (ALLODERM® Regenerative Tissue Matrix (LifeCell)); fetal bovine dermis (PRIMATRIX™ Dermal Repair Scaffold (TEI Biosciences)); porcine urinary bladder (MATRISTEMTM Extracellular Matrix Wound Sheet (Medline Industries, Inc.)); and porcine small intestinal submucosa (OASIS® Wound Matrix (Healthpoint Ltd.)).

As noted above, when healing, wounds go through three main stages; inflammation, proliferation and remodeling. In the inflammatory stage the body secretes proteases to the wound to remove damaged tissue and debris from the wound. In some cases when skin substitutes, such as an extracellular matrix, are inserted into the wound, proteases will attack the skin substitute and break it down as if the skin substitute were damaged tissue or debris. In other cases the skin substitutes, such as the extracellular matrix functions as intended, with cellular in-growth and provide shelter to the proliferating new cells.

Clinicians using matrix products typically inspect the wound 1-3 days after initial application of a matrix product to the wound bed. A significant problem identified by the inventors is that clinicians and medical practitioners are unable to easily and/or accurately distinguishing between a degraded skin substitute that has turned into slough and pus or skin substitute, such as a matrix, that has become wet and is being penetrated by cellular ingrowth. The inventors have found that being able to distinguish between a degraded skin substitute and a skin substitute within a wound that is properly healing, that is, for example in the case of a matrix skin substitute, that is being penetrated by cellular ingrowth, is key in efficient healing of the wound. If the added skin substitute material has become degraded, or a portion thereof has become degraded, the degraded skin substitute material must be removed, and the wound must be washed to remove the slough and pus that is often included with a degraded skin substitute. After washing and removal of the slough and pus, a new treatment of matrix material can be applied to the wound. However, if it is determined that the added matrix material is being penetrated by cellular ingrowth, as is intended, the matrix is left in place and monitoring of the matrix material continues as the wound properly heals.

For example, when healing wounds using fish-skin-derived cellular scaffold product (for example, as disclosed in U.S. Pat. No. 8,613,957, granted Dec. 24, 2013), the inventors have found that clinicians and care providers unwittingly mistake or otherwise struggle to distinguish between the wound healing scaffold and infection. This may be due, at least in part, to the color and/or odor associated with the wound healing scaffold once it starts to break down and integrate into the surrounding tissue. It can sometimes have a similar color as infected tissue (e.g., a purulent infection) and may also be mildly odoriferous, which some may interpret to be a similar odor as infected tissue.

Thus, the inventors have further identified that without an efficient and effective means of determining whether the skin substitute is being penetrated by cellular ingrowth, unnecessary removal or changes of dressing are required to inspect the wound, wound exposure, and unnecessary reapplications of the skin substitute made, which hinder proper healing of the wound.

Additionally, infection is a major challenge in wound healing and management. For example, in the case of combat wounds, infection determines the morbidity and mortality of injured service members on the battlefield. Infection accounts for one-third of total casualties, prolonged treatments, and an increased risk of amputation. Because of the distinct mechanisms of injury and the austere environment, combat wounds are prone to contamination, making treatment more difficult. An early sign of infection is bacterial imbalance within the wound. Common pathogens found in the wound at an early stage include both gram-positive (G+) and gram-negative (G−) strains. In the event of an infection, an emergence of gram-negative bacteria and multi-drug resistant (MDR) organisms are observed. There is a great need for an effective and immediate intervention to lower the risk of infection to benefit soldiers and act as a force multiplier in combat zones.

Accordingly, the inventors have further identified the problem that in addition to providing a means to determine whether a skin substitute is being penetrated by cellular ingrowth, the skin substitute itself would reduce infection or make infection less likely to occur.

### SUMMARY

To address the problems noted above, the inventors herein disclose ingrowth- indicatory wound treatments, comprising a skin substitute and a coloring agent added to the skin substitute. The coloring agent is a biocompatible coloring agent that degrades upon protease attack within a treated wound.

Further, wound treatment methods are provided comprising providing a tissue-regenerating wound treatment composition, applying the tissue-regenerating wound treatment composition to a wound bed, and determining whether the skin substitute has been degraded by protease attack within the wound by determining a change in color of the coloring agent. The tissue-regenerating wound treatment comprises a skin substitute and a coloring agent added to the skin substitute. The coloring agent is a biocompatible coloring agent that degrades upon protease attack within a treated wound.

Methods of producing a tissue-regenerating wound treatment are provided, comprising providing a skin substitute and adding a coloring agent to the skin substitute. The coloring agent is a biocompatible coloring agent that degrades upon protease attack within a treated wound.

According to embodiments described herein, the skin substitute is a biological skin substitute, or synthetic substitute, or a hybrid of biological and synthetic skin substitutes.

According to one or more embodiments, the skin substitute is an autologous skin graft, a syngeneic skin graft, an allogeneic skin graft, a xenogeneic skin graft, or a synthetic skin graft.

According to one or more embodiments, the skin substitute includes a scaffold material.

According to one or more embodiments, the skin substitute includes a scaffold material that includes an extracellular matrix product.

According to one or more embodiments, the extracellular matrix product is in the form of particles, or a sheet, or a mesh.

According to one or more embodiments, the skin substitute is a scaffold material comprising intact decellularized fish skin, and the intact decellularized fish skin comprises extracellular matrix material.

According to one or more embodiments, the coloring agent includes a thiazine dye, or a triarylmethane dye, or a combination of a thiazine dye and a triarylmethane dye.

According to one or more embodiments, the coloring agent includes methylene blue (MB), or gentian violet (GV), or a combination of methylene blue (MB) and gentian violet (GV).

According to one or more embodiments, the skin substitute is lyophilized, wherein the coloring agent is added to the skin substitute before lyophilization or re-lyophilization of the skin substitute.

According to one or more embodiments, the coloring agent is added to the skin substitute by dyeing the skin substitute with a dye solution containing 0.01 wt % to 0.0001 wt % of the coloring agent in deionized water or in a phosphate-buffered saline solution.

According to one or more embodiments, the coloring agent is characterized by having one or more of antibiotic, antiseptic, antimicrobial, antiviral, antifungal, antiparasitics, anti-inflammatory, or antioxidant properties.

According to one or more embodiments, the tissue-regenerating wound treatment further comprises an added active agent that includes one or more of antibiotics, antiseptics, antimicrobial agents, antivirals, antifungals, antiparasitics, anti-inflammatory agents, antioxidants, drugs, proteins, peptides, or combinations thereof.

According to one or more embodiments, the coloring agent does not cause a permanent coloring of the wound upon healing.

As described herein, the tissue-regenerating treatments and methods disclosed herein provide healing progress monitoring of a wound and provide an accurate, efficient, and effective means to distinguish between a degraded applied skin substitute that has been applied to a wound and a skin substitute that is being successfully penetrated by cellular ingrowth. This enables the clinician or medical practitioner to easily to distinguish between (1) having to wash the wound and remove an unsuccessfully applied skin substitute with accompanying slough and pus or (2) leaving the applied skin substitute in place, a coloring agent is added to the skin substitute, the coloring agent being of the nature of degrading by the protease attack within a treated wound. That is, a coloring agent is added to the skin substitute, for example, in the manufacturing stage, and the color of the coloring agent is characterized by being changed, removed, or broken down by one or more proteases in the treated wound.

This summary is provided to introduce a selection of concepts in a simplified form that are further described below in the detailed description. This summary is not intended to identify key features or essential features of the claimed subject matter, nor is it intended to be used as an indication of the scope of the claimed subject matter.

Additional features and advantages of the disclosure will be set forth in the description that follows, and in part will be obvious from the description, or may be learned by the practice of the disclosure. The features and advantages of the disclosure may be realized and obtained by means of the instruments and combinations particularly pointed out in the appended claims. These and other features of the present disclosure will become more fully apparent from the following description and appended claims or may be learned by the practice of the disclosure as set forth hereinafter.

These and other present features, aspects, and advantages of the present disclosure will become better understood regarding the following description, appended claims, and accompanying drawings.

### BRIEF DESCRIPTION OF THE DRAWINGS

FIGS. 1A, 1B, 1C, 1D, 1E, and 1F illustrate embodiments of skin substates according to the present disclosure.

FIGS. 2A, 2B, 2C, 2D, and 2E illustrate embodiments of skin substates according to the present disclosure in the form of decellularized fish skin.

FIG. 3 illustrates a colored skin substitute according to an embodiment of the disclosure.

FIGS. 4A, 4B, and 4C illustrate various colored skin substitutes according to embodiments of the disclosure.

FIG. 5 illustrates a colored skin substitute according to an embodiment of the disclosure.

FIGS. 6A, 6B, 6C, and 6D illustrate various mordanted and colored skin substitutes according to embodiments of the disclosure.

FIG. 7 illustrates various colored skin substitutes dyed under pH grading according to an embodiment of the disclosure.

FIG. 8 illustrate colored skin substitutes according to embodiments of the disclosure.

FIGS. 9A and 9B illustrate colored skin substitutes according to embodiments of the disclosure before and after exposure to collagenase.

FIGS. 10A and 10B show before and after treatments of a wound of a patient according to methods and embodiments of the disclosure.

FIGS. 11A and 11B show before and after treatments of a wound of a patient according to methods and embodiments of the disclosure.

FIGS. 12A, 12B, 12C, 12D, 12E, 12F, 12G, 12H, 12I, 12J, 12K, 12L, and 12M, show before, during, and after treatments of wounds of a patient according to methods and embodiments of the disclosure.

FIG. 13 shows an examplary method of treatment of a wound using a tissue-regenerating wound treatment according to embodiments of the disclosure.

FIGS. 14A, 14B, and 14C illustrate results of bacterial inhibition/reduction assays according to embodiments of the disclosure.

FIGS. 15A, 15B, and 15C illustrate comparisons of skin grafts according to embodiments of the present disclosure.

FIG. 16 shows an embodiment of a crosslinked, dyed scaffold material.

FIG. 17 shows another embodiment of a crosslinked, dyed scaffold material.

FIG. 18 shows another embodiment of a crosslinked, dyed scaffold material.

FIGS. 19A and 19B show comparisons of color fastness of embodiments of crosslinked, dyed scaffold materials.

FIGS. 20A, 20B, 20C, and 20D show comparisons of color fastness of other embodiments of crosslinked, dyed scaffold materials.

The drawing figures are not necessarily drawn to scale. Instead, they are drawn to provide a better understanding of the components and are not intended to be limiting in scope but to provide exemplary illustrations. The figures illustrate exemplary configurations of a wound treatment and features and sub-components thereof according to the present disclosure.

### DETAILED DESCRIPTION

A better understanding of different embodiments of the disclosure may be had from the following description read with the accompanying drawings in which like reference characters refer to like elements.

While the disclosure is susceptible to various modifications and alternative constructions, certain illustrative embodiments are in the drawings described below. It should be understood, however, there is no intention to limit the disclosure to the specific embodiments disclosed, but on the contrary, the intention covers all modifications, alternative constructions, combinations, and equivalents falling within the spirit and scope of the disclosure.

The references used are provided merely for convenience and hence do not define the sphere of protection or the embodiments.

It will be understood that unless a term is expressly defined in this application to possess a described meaning, there is no intent to limit the meaning of such term, either expressly or indirectly, beyond its plain or ordinary meaning.

Any element in a claim that does not explicitly state “means for” performing a specified function, or “step for” performing a specific function should not to be interpreted as a “means” or “step” clause as specified in 35 U.S.C. § 112.

Skin Substitute(s)

As described above, many different types of skin substitutes may be used to aid in the healing process of a wound and to more quickly restore at least some of the functions of healthy skin. Skin substitutes may be considered broadly as a group of elements or materials that enable the temporary or permanent occlusion of a wound. Skin substitutes can generally be divided into biological skin substitutes, synthetic skin substitutes, or a hybrid skin substitute that includes biological and synthetic skin substitutes.

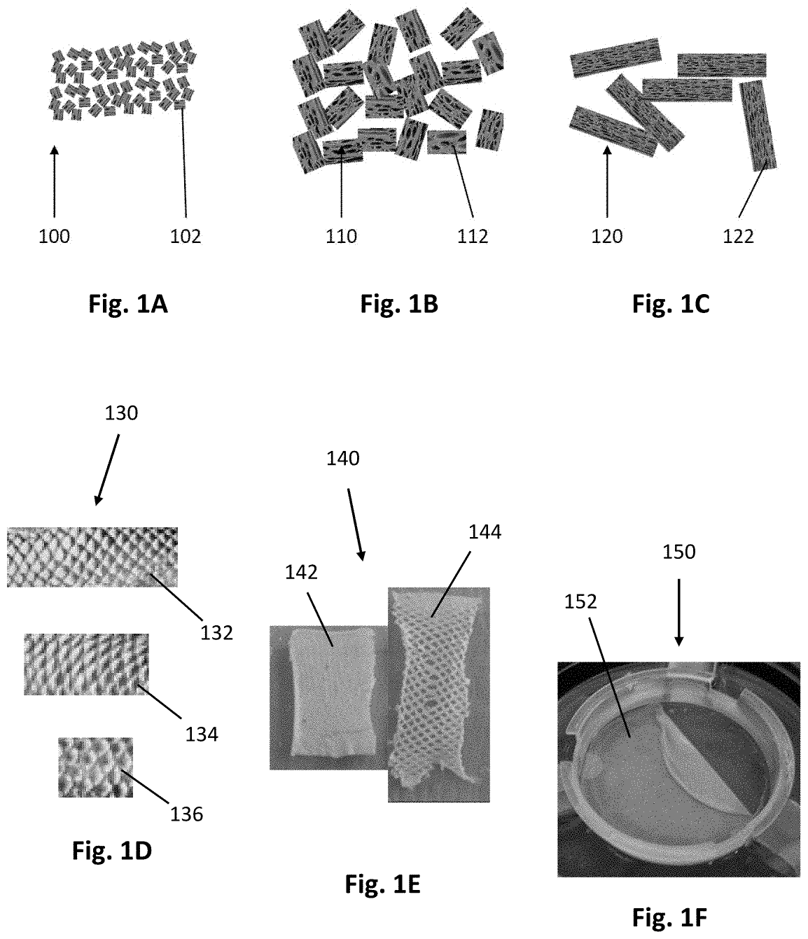

Examples of such skin substitutes are shown in FIGS. 1A to 1F.

FIG. 1A shows an example of skin substitute 100 of a wound treatment according to an embodiment comprising shredded, decellularized fish skin particles 102 in a first size. FIG. 1B shows an example of skin substitute 110 of a wound treatment according to an embodiment comprising shredded, decellularized fish skin particles 112 in a second size. FIG. 1C shows an example of skin substitute 120 of a wound treatment according to an embodiment comprising shredded, decellularized fish skin particles 122 in a third size. In the embodiments of FIGS. 1A, 1B, and 1C, the decellularized fish skin scaffold material is biocompatible and thus can be integrated by the host. An example of such a commercially available decellularized fish skin scaffold material is Omega3™ Wound by Kerecis, which is made from the minimally processed skin of wild-caught Atlantic cod, as described in U.S. Pat. No. 8,613,957.

Other examples of applicable skin substitutes are shown in FIGS. 1D, 1E, and 1F. FIG. 1D shows an example of skin substitute 130 produced from a processed fish skin of tilapia. A tilapia-based skin graft may be provided in various sizes including a large skin graft 132, a medium skin graft 134, and a small skin graft 136. FIG. 1E shows example of porcine skin grafts 140, which include a non-meshed porcine skin graft 142 and a meshed porcine skin graft 144. An additional example of a skin substitute is shown in FIG. 1F, which shows a synthetic skin substitute 150, which in this case is a bioengineered skin substitute formed of a bilayer tissue 152. In this non-limiting example, the bilayer tissue 152 of synthetic skin substitute 150 dermal layer is type I bovine collagen gel seeded with living human neonatal fibroblasts. The epidermis is neonatal keratinocytes. The cells actively secrete growth factors, cytokines, and extracellular matrix (ECM) proteins. A non-limiting example of such a synthetic skin substitute may include Apligraf™, which may used to treat diabetic foot ulcers and venous leg ulcers.

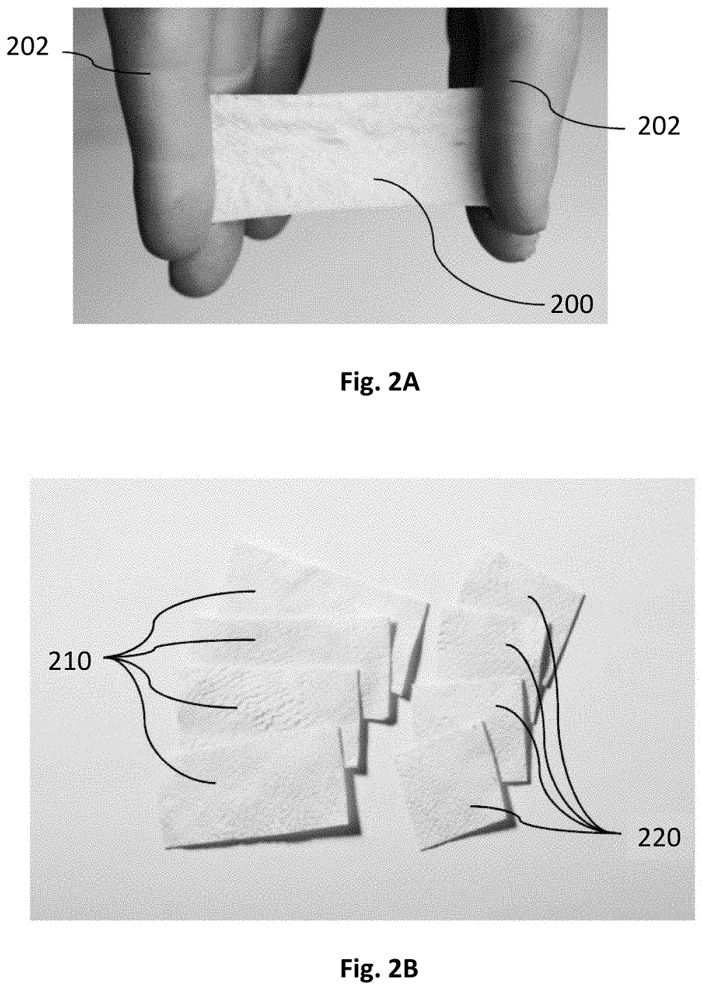

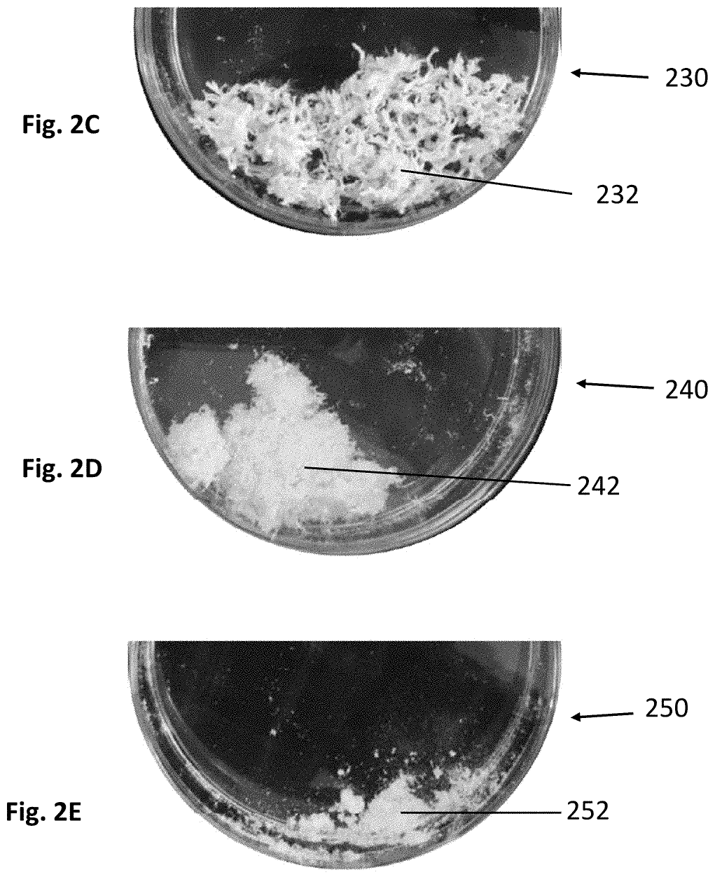

Referring now to FIGS. 2A and 2B, illustrated are exemplary embodiments of pieces of decellularized fish skin 200, 210. An exemplary section of decellularized fish skin 200, made as described in U.S. Pat. No. 8,613,957, is illustrated in FIG. 2A with the size thereof given context by the user's gloved hands 202. The size of decellularized fish skin 200 is of course non-limiting, and may be produced or provided, or trimmed to fit the size and shape of the wound to be treated. Further, although the shown decellularized fish skin is a non-meshed fish skin, a meshed decellularized fish skin may also be used.

It should be appreciated that the decellularized fish skin can be particalized, comminuted, or otherwise processed into various sizes and shapes. As shown in FIG. 2B, a plurality of decellularized fish skin sheets 210 can be sized and shaped similar to the decellularized fish skin 200 of FIG. 2A (e.g., rectangular) or they can have more uniform dimensions (e.g., squares), such as the decellularized fish skin sheets 220 illustrated in FIGS. 2B.

The decellularized fish skin scaffold 210, 220 depicted in FIGS. 2A and 2B is substantially rigid and inelastic in lyophilized form. The decellularized fish skin scaffold can be treated with one or more enzymes that act to increase its ductility and/or elasticity. In some embodiments, the enzymes act by cleaving interconnected extracellular matrix components without substantially impacting the salubrious properties important for wound preservation and/or stabilization. In some embodiments, the enzymes cleave covalent bonds within and/or between elastins, proteoglycans, collagens, or other extracellular matrix materials, but the modified decellularized fish skin retains a substantial portion of the extracellular matrix contents, even if partially removed from its natural three-dimensional structure.

In some embodiments, the enzyme treatment negatively impacts the use of the modified decellularized fish skin as a scaffold material. It should be appreciated, however, that loss of function as a scaffold material, surprisingly, does not appreciably impact the use of decellularized fish skin as a wound preservation and stabilization material. Thus, the ductility and/or elasticity of the material may be increased while maintaining the composition of the extracellular components, and even though this may negatively affect the use of the material as a scaffold for wound healing, the modified decellularized fish skin can nonetheless act as a wound preservation/stabilization material.

The decellularized fish skin scaffold can be comminuted and provided in particle form. It should be appreciated that the size of individual comminuted particles may vary, depending on the type and/or manner of comminution. For example, decellularized fish skin particles can be created through a jet milling process designed to output particles below a specified size. In some embodiments, decellularized fish skin is cut, chopped, or ground into particles, which may be done in a measured fashion to create uniform particles or roughly performed, thereby generating a variety of different sized particles.

FIG. 2C illustrates an exemplary depiction of large particles 232 of particalized or comminuted decellularized fish skin 230 resulting from grinding a sheet of decellularized fish skin scaffold material with a grinder, for example, a hemp grinder. FIG. 2D illustrates an exemplary depiction of threaded, cotton-like fibers 242 of particalized or comminuted decellularized fish skin 240 resulting from grinding a sheet of decellularized fish skin scaffold material with a grinder in accordance with embodiments of the present disclosure. FIG. 2E is an exemplary depiction of small, powder-like particles 252 of comminuted decellularized fish skin 250 resulting from grinding a sheet of decellularized fish skin scaffold material with a grinder, for example, a hemp grinder.

In embodiments, the wound treatment is or comprises particularized, particularly shredded, decellularized fish skin particles of at least one predetermined size. The particularized, i.e. shredded, decellularized fish skin particles are configured to provide a scaffold material for supporting cell migration, adherence, proliferation, and differentiation for facilitating the repair and/or replacement of tissue, as described in U.S. Pat. No. 8,613,957, granted on Dec. 24, 2013, the application of which was filed Oct. 6, 2010, the contents of which are incorporated by reference herein in its entirety.

The extracellular matrix (ECM) of vertebrates is a complex structural entity surrounding and supporting cells. ECM is composed of complex mixtures of structural proteins, the most abundant of which is collagen, and other specialized proteins and proteoglycans. The scaffold material described herein is a largely intact acellular scaffold of natural biological ECM components from fish skin. The scaffold can also comprise naturally occurring lipids from the fish skin. The native three-dimensional structure, composition, and function of the dermal ECM is essentially unaltered, and provides a scaffold to support cell migration, adherence, proliferation, and differentiation, thus facilitating the repair and/or replacement of tissue.

A scaffold material in accordance with this invention is obtained from intact fish skin. Any species of fish, including bony or cartilaginous fish, can be used as the source of the fish skin. For example, the source can be round fish like cod, haddock and catfish; flatfish, like halibut, plaice and sole; salmonids like salmon and trout; scombridaes like tuna; or small fish like herring, anchovies, mackerel and sardines. In certain embodiments the fish skin is obtained from cold-water oily fish and/or fish known to contain high amounts of omega-3 oil. Examples of fish high in omega-3 oil are salmon, pilchards, tuna, herring, cod, sardines, mackerel, sable fish, smelts, whitefish, hoki fish, and some varieties of trout.

The fish skin is removed from the fish before processing. If the fish skin is from a species of fish that has scales, the fish skin should be de-scaled so that a substantial portion of the scales are removed or at least the hydroxyapatite removed from the scales. The phrase “a substantial portion of the scales are removed” or “substantially scale-free” means that at least 95%, preferably at least 99%, and more preferably 100% of the scales on the fish skin are removed. “Substantially scale free” fish skin can also refer to fish skin from a fish species without scales. The scales are either removed prior to all processing, with purely mechanical pressure (via, e.g., knife, shaking with abrasives, water pressure, a special scale removal device that uses the same mechanical force as knives or other pressure device, like polishing with ceramic or plastic) or after some chemical treatment (e.g. decellularization) and then with mechanical pressure in order to wash the scales away. If the fish skin is first treated chemically and/or enzymatically (e.g. treatment with TRITON® X-100), the mechanical pressure generally needs to be gentle since the skin is more vulnerable to tearing after decellularization. The scales can be removed in more than one step, for example partial removal prior to decellularization followed by further removal during and/or after decellularization. Alternatively the scales can be removed by chemical treatment alone.

After the scales have been removed, the fish skin is optionally frozen prior to decellularization. The fish skin can be frozen quickly by incubating the skin in liquid nitrogen or using other special freezing equipment that can freeze the skin to −70° C. or lower, in order to preserve the collagen structure of the scaffold. Alternatively, the fish skin can be frozen in a conventional type of freezer that would be typically found in a fish factory. The freezing process may lyse or partially lyse the cells comprising the intact fish skin, and help facilitate decellularization of the fish skin. If the fish skin has been frozen, it can later be thawed for further processing.

Whether or not the fish skin was frozen, it can be washed with a buffer solution prior to further processing. For example, the fish skin can be washed 1-3 times with a buffer solution optionally containing one or more antioxidants (e.g. ascorbic acid (such as 50 mM ascorbic acid), Vitamins A, C, E, and beta carotene), antibiotics (e.g., streptomycin and penicillin), proteases (e.g. dispase II) and protease inhibitors (e.g. antipain, aprotinin, benzamidine, bestatin, DFP, EDTA, EGTA, leupeptin, pepstatin, phosphoramidon, and PMSF) to facilitate disinfection and stabilization of the fish skin. The buffer solution can be at a pH of at least 5.5, such as 6.0, 6.5, 7.0, 7.5, 8.0, 8.5, 9.0, 9.5, 10.0 or more. In certain embodiments the pH is between 7.0 and 9.0, e.g. between 7.5 and 8.5. The buffer solution can also be used as a medium in which the fish skin can be stored for a few days up to a few weeks or more. In certain embodiments the fish skin is stored in the buffer solution at a temperature of about 4° C.

After freezing and/or washing and/or storage in a buffer solution, the fish skin is treated with one or more decellularizing solutions to remove cellular material, including antigenic material, from the fish skin with minimal to no damage to the mechanical and structural integrity and biological activity of the naturally occurring extracellular matrix.

The terms “extracellular matrix” or “ECM” as used herein refer to the non-cellular tissue material present within the fish skin that provides structural support to the skin cells in addition to performing various other important functions. The ECM described herein does not necessarily include matrix material that has been constituted or re-formed entirely from extracted, purified, or separated ECM components (e.g. collagen). But in some embodiments, an ECM used as a skin substitute may include matrix material that has been constituted or re-formed entirely from extracted, purified, or separated ECM components (e.g. collagen).

The terms “acellular,” “decellularized,” “decellularized fish skin,” and the like as used herein refer to a fish skin from which a substantial amount of cellular and nucleic acid content has been removed leaving a complex three-dimensional interstitial structure of ECM. In embodiments, “decellularized fish skin” may further entail fish skin which, in addition to the complex three-dimensional interstitial structure of ECM absent a substantial amount of cellular and nucleic acid content, includes omega 3 polyunsaturated fatty acids (PUFAs).

“Decellularizing agents” are those agents that are effective in removing a substantial amount of cellular and nucleic acid content from the ECM. The ECM is “decellularized” or “substantially free” of cellular and nucleic acid content (i.e. a “substantial amount” has been removed) when at least 50% of the viable and non-viable nucleic acids and other cellular material have been removed from the ECM. In certain embodiments, about 55%, 60%, 65%, 70%, 75%, 80%, 85%, 90%, 91%, 92%, 93%, 94%, 95%, 96%, 97%, 98%, 99%, 99.5%, or 100% of the viable and non-viable nucleic acids and cellular material are removed. Decellularization can be verified by, for example, testing the treated fish skin for DNA content. Removal of the nucleic acids from the ECM can be determined, for example, by histological examination of the ECM, and/or by a biochemical assay such as the PICOGREEN® assay, diphenylamine assay, or by PCR.

Decellularization disrupts the cell membranes and releases cellular content. Decellularizing may involve one or more physical treatments, one or more chemical treatments, one or more enzymatic treatments, or any combination thereof. Examples of physical treatments are sonication, mechanical agitation, mechanical massage, mechanical pressure, and freeze/thawing. Examples of chemical decellularizing agents are ionic salts (e.g. sodium azide), bases, acids, detergents (e.g. non-ionic and ionic detergents), oxidizing agents (e.g. hydrogen peroxide and peroxy acids), hypotonic solutions, hypertonic solutions, chelating agents (e.g. EDTA and EGTA), organic solvents (e.g. tri(n-butyl)-phosphate), ascorbic acid, methionine, cysteine, maleic acid, and polymers that bind to DNA (e.g. Poly-L-lysine, polyethylimine (PEI), and polyamindoamine (PAMAM)). Non-ionic detergents include 4-(1,1,3,3-Tetramethylbutyl)phenyl-polyethylene glycol, t-Octylphenoxypolyethoxyethanol, Polyethylene glycol tert-octylphenyl ether (TRITON® X-100) (Dow Chemical Co.). Ionic detergents include sodium dodecyl sulfate (SDS), sodium deoxycholate, TRITON® X-200, and zwitterionic detergents (e.g. CHAPS). Other suitable decullularizing detergents include polyoxyethylene (20) sorbitan mono-oleate and polyoxyethylene (80) sorbitan mono-oleate (Tween 20 and 80), 3-[(3-chloramidopropyl)-dimethylammino]-1-propane-sulfonate, octyl-glucoside and sodium dodecyl sulfate. Examples of enzymatic decellularizing agents are proteases, endonucleases, and exonucleases. Proteases include serine proteases (e.g. trypsin), threonine proteases, cysteine proteases, aspartate proteases, metalloproteases (e.g. thermolysin), and glutamic acid proteases. Decellularization is generally carried out at a pH of at least 5.5, such as 6.0, 6.5, 7.0, 7.5, 8.0, 8.5, 9.0, 9.5, 10.0 or more. In certain embodiments the pH is between 7.0 and 9.0, e.g. between 7.5 and 8.5.

An example of a decellularization step is incubating the fish skin in a solution comprising 1 M NaCl, 2% deoxycholic acid, 0.02% sodium azide and 500 ppm streptomycin. In another example, the fish skin is incubated with a first decellularizing solution comprising a protease (e.g., 2.5 U/mL dispase II) and other components (e.g., 0.02% sodium azide). The first decellularizing solution is poured off and the fish skin is then treated with a second decellularizing solution such as a solution comprising a detergent (e.g., 0.5% TRITON® X-100) and other components (e.g. 0.02% sodium azide). In another example, the fish skin is first treated with a decellularizing solution comprising detergent (e.g. 0.5% TRITON® X-100) with other components (e.g. 0.02% EDTA, sodium azide, and/or deoxiholic acid), and then incubated in a second decellularizing solution comprising a detergent such as SDS.

The fish skin may or may not be incubated under shaking. The decellularizing step(s) can be repeated as needed by pouring off any remaining decellularizing solution, optionally washing the fish skin with a buffer solution (e.g. Hank's Balanced Salt Solution), and then treating the fish skin again with another step of decellularization. Once a sufficient amount of cell material has been removed, the decellularizing solution can be removed (e.g., by aspiration or by gently pouring out the solution).

After decellularization, the fish skin can optionally be washed with water, buffer solution, and/or salt solution. Examples of suitable washing solutions include Dulbecco's phosphate buffered saline (DPBS), Hank's balanced salt solution (HBSS), Medium 199 (M199, SAFC Biosciences, Inc.) and/or L-glutamine. Washing step(s) are generally carried out at a pH of at least 5.5, such as 6.0, 6.5, 7.0, 7.5, 8.0, 8.5, 9.0, 9.5, 10.0 or more. In certain embodiments the pH is between 7.0 and 9.0, e.g. between 7.5 and 8.5.

The fish skin can optionally be bleached to improve the appearance of the final product. Bleaching can be carried out before, after, and/or concurrently with decellularization. For example, one or more bleaching agent can be incorporated into one or more of the decellularization solution(s) and/or into one or more buffer solution(s). Examples of bleaching agents include sodium sulfite, hydrogen peroxide, ammonium persulfate, potassium persulfate, and sodium persulfate. In certain embodiments, if a strong bleaching agent like persulfate(s) are used, bleaching and decellularization can be combined in a single step comprising incubating the fish skin in a mixture of one or more bleaching agents, thickeners, and peroxide sources. For example, a dry bleaching mixture can be prepared (see, e.g., the “bleaching mixtures” described in Example 5), followed by the addition of water, hydrogen peroxide, or a combination thereof to the dry mixture to form a bleaching solution that may also be sufficient for decellularization. The bleaching agents (e.g. sodium sulfite, hydrogen peroxide, ammonium persulfate, potassium persulfate, and sodium persulfate) should be about 40-60% w/w of the dry mixture. A combination of EDTA and persulfates may be added to the mixture to accelerate bleaching as well as decellularization.

In certain embodiments the concentration of EDTA in the dry mixture is about 0.25-5% w/w. Hydrogen peroxide can be about 15-25% of the mixture; the peroxide source can be sodium percarbonate and potassium percarbonate. Sodium phosphate perhydrate and sodium carbonate or magnesium metasilicate and silicium silicate can also be used as a peroxide source. The dry mixture can also include silica and hydrated silica, at for example 1-10% w/w, and optionally one or more stearate (e.g. ammonium stearate, sodium stearate, and/or magnesium stearate). In addition the dry mixture can optionally include thickeners, such as hydroxypropyl methylcellulose, hydroxyethylcellulose, algin (i.e. alginate), organic gums (e.g. cellulose, xanthan gum) sodium metasilicate, and combinations thereof to increase the viscosity of the bleaching/decellularization solution and protect protein fibers from damage. Bleaching, and/or bleaching plus decellularization, is generally carried out at a pH of at least 5.5, such as 6.0, 6.5, 7.0, 7.5, 8.0, 8.5, 9.0, 9.5, 10.0 or more. In certain embodiments the pH is between 7.0 and 9.0, e.g. between 7.5 and 8.5. After bleaching and/or bleaching plus decellularization, the fish skin is optionally washed with a solution comprising L-glutamine under the pH conditions described above.

In certain embodiments, the fish skin is treated with a digestion enzyme. Similar to bleaching, digestion can be carried out before, after, and/or concurrently with decellularization. Suitable enzymes include proteases, for example serine proteases, threonine proteases, cysteine proteases, aspartate proteases, metalloproteases, and glutamic acid proteases. In certain embodiments the digestion enzyme is a serine protease such as trypsin. The digestion enzyme can be an enzyme that functions in an alkaline environment, limits cross-linking within the ECM, and softens the fish skin. Digestion is generally carried out at a pH of at least 5.5, such as 6.0, 6.5, 7.0, 7.5, 8.0, 8.5, 9.0, 9.5, 10.0 or more. In certain embodiments the pH is between 7.0 and 9.0, e.g. between 7.5 and 8.5.

The decellularized fish skin can optionally be cryopreserved. Cryopreservation can involve immersing the fish skin in a cryoprotectant solution prior to freezing. The cryoprotectant solution generally comprises an appropriate buffer, one or more cryoprotectants, and optionally a solvent, e.g. an organic solvent which in combination with water undergoes minimal expansion and contraction. Examples of cryoprotectants include sucrose, raffinose, dextran, trehalose, dimethylacetamide, eimethylsulfoxide, ethylene glycol, glycerol, propylene glycol, 2-Methyl-2.4-pantandial, certain antifreeze proteins and peptides, and combinations thereof Alternatively, if the decellularized fish skin is fast-frozen (flash-frozen) prior to sublimation in order to minimize ice crystals formed during the freezing step, the skins can optionally be frozen in a buffer solution that does not include cryoprotectants. Cryopreservation is generally carried out at a pH of at least 5.5, such as 6.0, 6.5, 7.0, 7.5, 8.0, 8.5, 9.0, 9.5, 10.0 or more. In certain embodiments the pH is between 7.0 and 9.0, e.g. between 7.5 and 8.5.

The decellularized fish skin can be packaged inside a sterile container, such as a glass vial or a pouch. In one embodiment, a TYVEK® pouch is used. For example, the fish skin can be incubated in a cryoprotectant solution, packaged in a TYVEK® pouch and then placed into a freeze dryer and frozen at a rate which is compatible with the cryoprotectant.

The decellularized fish skin can be lyophilized, i.e. frozen at a low temperature and under vacuum conditions so that water is removed sequentially from each ice crystal phase without ice re-crystallization. During lyophilization, water is generally removed first via sublimation and then via desorption if necessary. Another method of removing excess water after processing and before sterilization is vacuum pressing.

In certain embodiments, the decellularized fish skin is sterilized before and/or after being frozen. Sterilization methods are well known in the art. For example, the decellularized fish skin can be placed in an ethylene oxide chamber and treated with suitable cycles of ethylene oxide. Other sterilization methods include sterilizing with ozone, carbon dioxide, gaseous formaldehyde or radiation (e.g. gamma radiation, X-rays, electron beam processing, and subatomic particles).

As an alternative to or in addition to freezing, freeze-drying and/or vacuum pressing of water, the decellularized fish skin can be preserved in a non-aqueous solution such as alcohol.

The resulting product (scaffold material) is a sterile, collagen-based matrix that possesses properties that may facilitate the regeneration, repair and/or replacement of tissue (e.g., repair, regeneration, and/or growth of endogenous tissue). The term “scaffold material” in the context of fish skins refers to material comprising fish skin that has been decellularized and optionally bleached, digested, lyophilized, etc. as discussed above. The scaffold material can provide an intact scaffold for support of endothelial and/or epithelial cells, can be integrated by the host, is biocompatible, does not significantly calcify, and can be stored and transported at ambient temperatures. The phrase “integrated by the host” means herein that the cells and tissues of the patient being treated with the scaffold material can grow into the scaffold material and that the scaffold material is actually integrated/absorbed into the body of the patient. The term “biocompatible” refers to a material that is substantially non-toxic in the in vivo environment of its intended use, and that is not substantially rejected by the patient's physiological system (i.e., is non-antigenic).

This can be gauged by the ability of a material to pass the biocompatibility tests set forth in International Standards Organization (ISO) Standard No. 10993 and/or the U.S. Pharmacopeia (USP) 23 and/or the U.S. Food and Drug Administration (FDA) blue book memorandum No. G95-1, entitled “Use of International Standard ISO-10993, Biological Evaluation of Medical Devices Part 1: Evaluation and Testing.” Typically, these tests measure a material's toxicity, infectivity, pyrogenicity, irritation potential, reactivity, hemolytic activity, carcinogenicity and/or immunogenicity. A biocompatible structure or material, when introduced into a majority of patients, will not cause a significantly adverse, long-lived or escalating biological reaction or response, and is distinguished from a mild, transient inflammation which typically accompanies surgery or implantation of foreign objects into a living organism.

The scaffold material contains proteins from the extracellular matrix (ECM) of the fish skin. The ECM components in the scaffold material can include, for example, structural proteins; adhesive glycoproteins; proteoglycans; non-proteoglycan polysaccharides; and matricellular proteins. Examples of structural proteins include collagens (the most abundant protein in the ECM), such as fibrillar collagens (types I, II, III, V, and XI); facit collagens (types IX, XII, and XIV), short chain collagens (types VIII and X), basement membrane collagen (type IV), and other collagens (types VI, VII, and XIII); elastin; and laminin. Examples of adhesive glycoproteins include fibronectin; tenascins; and thrombospondin. Examples of proteoglycans include heparin sulfate; chondroitin sulfate; and keratan sulfate. An example of a non-proteoglycan polysaccharide is hyaluronic acid. Matricellular proteins are a structurally diverse group of extracellular proteins that regulate cell function via interactions with cell-surface receptors, cytokines, growth factors, proteases, and the ECM. Examples include thrombospondins (TSPs) 1 and 2; tenascins; and SPARC (secreted protein, acidic and rich in cysteine).

In certain embodiments, decellularization (and other optional processing steps) does not remove all of the naturally occurring lipids from the lipid layer of the fish skin. Thus, the scaffold material can comprise one or more lipids from the fish skin, particularly from the lipid layer of the fish skin. For example, the scaffold material may include up to about 25% w/w lipids (of dry weight of the total scaffold material after lyophilization), such as 0.1%, 0.5%, 1%, 2%, 3%, 4%, 5%, 6% 7%, 8%, 9%, 10%, 11%, 12%, 13%, 14%, 15%, 16%, 17%, 18%, 19%, 20%, 21%, 22%, 23%, or 24% w/w lipids. The presence of lipids in the scaffold material can be verified, for example, by organic solvent extraction followed by chromatography. Examples of suitable organic solvents include acetone and chloroform.

The lipids in the scaffold material can include, for example, fatty acyls (i.e. fatty acids, their conjugates, and deriviates); glycerolipids; glycerophospholipids (i.e. phospholipids); sphingolipids; saccharolipids; polyketides; sterol lipids (i.e. sterols); certain fat-soluble vitamins; prenol lipids; and/or polyketides. Examples of fatty acyls include saturated fatty acids, such as polyunsaturated fatty acids; fatty esters; fatty amides; and eicosanoids. In certain embodiments the fatty acids include omega-3 fatty acids, such as eicosapentaenoic acid (EPA) and docosahexaenoic acid (DHA) (found in high concentration in fish oil). Other fatty acids found in fish oil include arachidic acid, gadoleic acid, arachidonic acid, butyric acid, caproic acid, caprylic acid, capric acid, lauric acid, myristic acid, palmitic acid, palmitoleic acid, stearic acid, oleic acid, vaccenic acid, linoleic acid, alpha-linolenic acid, gamma-linolenic acid, behenic acid, erucic acid, and lignoceric acid. Examples of glycerolipids include mono-, di-, and tri-substituted glycerols, such as monoacylglycerols, diacylglycerols, and triacylglycerols (i.e. monoglycerides, diglycerides, and triglycerides). Examples of glycerophospholipids include phosphatidylcholine; phosphatidylethanolamine; and phosphatidylserine. Examples of sphingolipids include phosphosphingolipids and glycosphingolipids. Examples of sterol lipids include cholesterol; steroids; and secosteroids (various forms of Vitamin D). Examples of prenol lipids include isoprenoids; carotenoids; and quinones and hydroquinones, such as Vitamins E and K.

The scaffold material can contain one or more added active agents (i.e. an agent that is added during or after processing of the scaffold material), such as antibiotics, antiseptics, antimicrobial agents, antivirals, antifungals, antiparasitics and anti-inflammatory agents. The active ingredient can be a compound or composition that facilitates wound care and/or tissue healing such as an antioxidant, or drug. It can also be a protein or proteins and/or other biologics. Antibiotics, antiseptics, and antimicrobial agents can be added in an amount sufficient to provide effective antimicrobial properties to the scaffold material. In certain embodiments, the antimicrobial agent is one or more antimicrobial metal, such as silver, gold, platinum, copper, zinc, or combinations thereof. For example, silver may be added to the scaffold material during processing in ionic, metal, elemental, and/or colloidal form. The silver may also be in combination with other antimicrobials. Anti-inflammatory agents can be added in an amount sufficient to reduce and/or inhibit inflammation at the wound or tissue area where the scaffold material is applied.

The scaffold material can be used in dried form. Alternatively, the scaffold material can be rehydrated prior to use. In certain embodiments, one or more scaffold materials are laminated together to form a thicker scaffold material.

Generally, the scaffold material is from about 0.1 to 4.0 mm thick (i.e. in cross-section), such as 0.5, 1.0, 1.5, 2.0, 2.5, 3.0 or 3.5 mm thick. The thickness can depend on a number of factors, including the species of fish used as the starting material, processing, lyophilization, and/or rehydration. Of course, the thickness is proportionately greater when the product comprises more than one layer of scaffold material.

The shredded, decellularized fish skin particles of wound treatment and method embodiments advantageously provide a sterile, collagen-based matrix that possesses properties that may facilitate the regeneration, repair, and/or growth of tissue, such as endogenous tissue, while being configured to be formed or added to a wound so as to better accommodate the geometry of a wound. In embodiments, the shredded, decellularized fish skin particles are configured to be packed into a wound, such as an undermined or tunneling wound, in ways that are not available using sheet-based materials. That is, the shredded, decellularized fish skin particles may be configured to promote integration, that is in which the cells and tissues of the patient being treated with the scaffold material can grow into the scaffold material and that the scaffold material is actually integrated/absorbed into the body of the patient.

Shredded, decellularized fish skin particles according to embodiments may, in embodiments, be configured for actively promoting wound healing as a physical scaffold for infiltrating cells involved in wound healing/repair, such as for cell ingrowth and neovascularization. The shredded, decellularized fish skin particles of wound treatment embodiments are configured to advantageously retain the three-dimensional (“3D”) structure of the decellularized fish skin with an Extracellular Matrix (“ECM”) that is recognizable, for instance, on a histology analysis. The dimensions of the shredded decellularized fish skin particles may additionally be configured so as to facilitate molding, packing, or otherwise applying the shredded decellularized fish skin particles into a wound cavity with greater precision than existing approaches to wound therapy.

In embodiments, the shredded, decellularized fish skin particles have a greatest dimension within a predetermined maximum size threshold and a minimum size threshold that is effective to preserve the matrix structure of the decellularized fish skin and to promote cellular regenerative ingrowth into a wound. That is, a greatest dimension, such as a greatest one of a length, width, and/or thickness of the shredded, decellularized fish skin particles, may be lower than a maximum size, such as 1 mm, and larger than a minimum size, such as a size at which the ECM is destroyed. In embodiments, the shredded, decellularized fish skin particles are obtained by providing a sheet of decellularized fish skin as described above and then shredding the sheet of decellularized fish skin and optionally sieving the shredded particles until the shredded, decellularized fish skin particles are within the predetermined minimum and maximum size thresholds.

The shredded, decellularized fish skin particles may further be configured to resist shear forces on account of their dimensions, thus allowing the shredded, decellularized fish skin particles to provide an improved wound treatment for patients who move or are moved between locations or settings, or during the normal course of activities by a patient, such as walking, during recovery.

The shredded, decellularized fish skin particles of embodiments may advantageously be topically applied to and/or implanted in a wound to provide a scaffold for cell ingrowth and neovascularization, with additional benefits including tissue scaffolding benefits, such as adhesion barrier, soft tissue repair, dehiscence prevention, and others.

Coloring agent(s)

Various examples of a coloring agent may be contemplated. In its broadest sense, the coloring agent contemplated herein is a coloring agent, or combination of color or coloring agents, that provides a color to the skin substitute that changes or loses color, based on changes of conditions within the wound during the healing process or changes to the skin substitute. In preferred embodiments, the coloring agent degrades upon attack by one or more proteases within the wound. With such a coloring agent, the color agent, upon degradation by the one or more proteases, loses its color. For example, the coloring agent may provide a blue or violet color to the skin substitute. But upon attack by one or more proteases within the wound after application of the wound treatment including the skin substitute and the coloring agent, the color of skin substitute of the wound treatment is also degraded or lost, whereby the color of the applied wound treatment changes to the original color of the skin substitute or to a different color. But the change of color of the coloring agent is not limited thereto, and may include a color shift upon a change of condition within the wound. For example, the color provided by the coloring agent may be triggered such that the original color of the skin substitute is not changed upon application or adding of the coloring agent. But a color change of the coloring agent may be triggered or causes by a change of conditions within the wound, thereby turning the skin substitute of the wound treatment to a new or different color than the original color of the skin substitute.

Dyes may be used as the coloring agent. A preferred example of a color agent is a thiazine dye, such as methylene blue (MB). The structure of methylene blue (MB) is provided below:

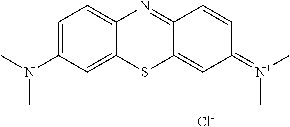

Methylene blue (MB) is also called methylthioninium chloride or basic blue 9. MB is a cationic thiazine dye used for a variety of applications, such as in fabric dyeing, medicine and research. It is used in the treatment of methemoglobinemia at a dose up to 2 mg/kg over few hours period.

Another embodiment of a coloring agent is a triarylmethane dye. An example of a preferred triarylmethane dye is gentian violet (GV), having the following structure:

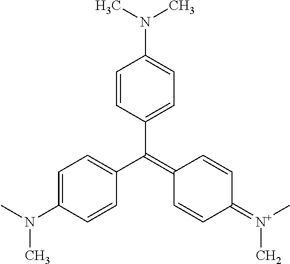

Gentian violet (GV) is also known as crystal violet, methyl violet 10B, or hexamethyl pararosaniline chloride. it's a triarylmethane dye commonly used for histological stain in Gram's method. Topical gentian violet (GV) is used to treat certain types of fungus infections inside the mouth (thrush) and of the skin.

Another embodiment of a coloring agent is brilliant blue FCF (BB-FCF), having the following structure:

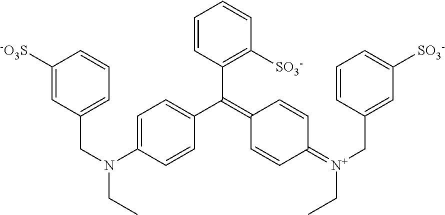

Brilliant blue FCF (BB-FCF), which is also called Blue No. 1, is a triarylmethane dye used primarily as a blue colorant for processed foods, medications, dietary supplements, and cosmetics. It is one of the oldest FDA-approved color additives and is generally considered nontoxic and safe.

Another embodiment of a coloring agent is indigo carmine (IC), having the following structure:

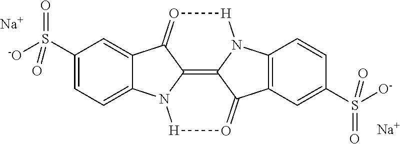

Indigo carmine (IC), which is also called food blue 1, is an organic salt derived from indigo by aromatic sulfonation, which renders the compound soluble in water. It is blue at pH below 11.4 and yellow above 13.0 and can additionally be used as a redox indicator turning yellow when reduced.

Other dye chemicals or dye mixes may be used and have been contemplated by the inventors, including the following.

Woad Powder (HUE-3023), which is a wool dye proposed by The Woolery, which has an INCI (International Nomenclature Cosmetic Ingredient) of woad extract. This is a commonly used dye chemical for yarn and clothes, and is usually used in coloring in an alkaline environment. Woad Powder may be considered a useful color agent due to the powder's nature of binding abilities to keratin protein.

Color additive D&C Green #5 Powder AN0725 is a made of natural sources and is used generally in cosmetic products. This color additive has an INCI name of Green No. 5. The powder is a water-based dye in a dry powder form. This may be chosen as a color agent due to its typical water-based cosmetic color in a powder form.

Color Additive Ultra Marine Blue H9-03R1 is used in cosmetic products, including eye makeup, soaps, lotions (but not for lip products). This color additive is based on natural sources and has an INCI name of Ultramarine Na6Al6Si6O24S4. This color additive may be is an oil dispersible pigment, will not dissolve in water or oil, and has a CAS-number: 57455-37-5. This is a strong dyer and may be chosen as a color agent because it is rated as very effective in cosmetics and because it will not dissolve in water or oil once put in the cosmetics. However, this color additive may include remains of undesirable substances, which must be considered.

Color additive Liquid FD&C blue #1 is used in cosmetic products, soaps, bath salts, and bath bombs. It is made of natural sources, and has an INCI name of Blue No. 1. This color additive may be provided in pre-mixed, water-based dye. This is a typical cosmetic water-soluble liquid dye.

Color additive Liquid D&C green #5 is also used in cosmetic products, soaps, bath salts, and bath bombs. It is made of natural sources, and has an INCI name of Green No. 5. This color additive may be provided in pre-mixed, water-based dye. This is a typical cosmetic water-soluble liquid dye.

Color additive Liquid D&C green #6 oil AM4299 is also used in cosmetic products, soaps, bath salts, and bath bombs. It is made of natural sources, and has an INCI name of Green No. 6 and Caprylic/Capric Triglyceride. This color additive is provided in pre-mixed oil-based liquid dye, blended, for example, in fractionated coconut oil for long shelf life. This dye may be considered for product that reacts better with an oil-based dye, which will then resist the washing step better and not dissolve in the hydration. However, it should noted that use of this dye should consider means to alleviate or address possible permanent coloring of the wound, which would case a tattooing effect to the patient.

Green Concentrated Food Coloring is a food coloring manufactured by Rayner. It has an INCI name of Water, tartrazine (E102) (1.87%), Brilliant blue FCF (E133) (0.13%), acetic acid. This may be considered as a coloring agent, as it provided in a pre-mixed water based liquid food dye mix. The dye mixture is considered to be harmless dye mixture.

Gamier natural Color, mahogany brown is a hair coloring produced by Gamier, having ingredients of aqua, deceth-3, alureth-12, cocamide mipa, oleth-30, ammonium hydroxide, deceth-5, glycerin, oleic acid, oleyl alcohol, hexadimethrine chloride 2, 4-diaminophonexyethanol HC1, p-aminophenol, m-aminophenol, ascorbic acid, hydroxyethylcellulose, sodium metabisulfite, ethanolamine, triticum vulgare germ oil, wheat germ oil, thioglycerin, polyquarternium-6, toluene-2,5-diamine, polyquarternium-67, 2-methyl-5-hydroxyethylaminophenol, ammonium thiolactate, simmondsia chinensis oil, jojoba seed oil, isopropanolamine, resorcinol, EDTA, parfum. This dye is commercially provided as a pre-mixed hair color kit. This dye is another embodiment of a coloring agent as it is formulated to bind with proteins and would be reactive with the collagen of a scaffold or a of a skin substitute.

ELEA, color and care, black, which is a hair coloring, produced by ELEA. This has having ingredients of Aqua, ceterayl alcohool, ammonia cetereth-20, cetrimonium chloride, cocamdopropyl betane, oleic acid, propylene glycol, PEG-40, hydrogenated castrol oil, p-phenylenediamine, 2,4-diaminophenoxyethanol, HC1, vitis vinferia seed oil sodium metabisulfite, erythorbic acid, parfum, coumarin, limonene, linalool, resorcinol, tetrasodium EDTA. This dye is another embodiment of a coloring agent as it is formulated to bind with proteins and would be reactive with the collagen of a scaffold or a of a skin substitute.

Although various examples and embodiments of coloring agents is provided here, this description of possible coloring agents is not and should not be considered to be exhaustive of all possible coloring agents, either as dyes or color additives.

Adding a Coloring Agent to a Skin Substitute

As a preferred embodiment, methylene blue (MB), gentian violet (GV), or a combination of methylene blue (MB) and gentian violet (GV) is used as a coloring agent to be added to a skin substitute.

In an embodiment that methylene blue (MB) and gentian violet (GV) are used in combination, methylene blue (MB) and gentian violet (GV) have been used together in equal weight ratios. But in other embodiments, methylene blue (MB) and gentian violet (GV) have been used together in unequal weight ratios. Other embodiments include any combination of two or more of these dyes, methylene blue (MB) and gentian violet (GV), and other dyes. Exemplary methods and embodiments will be described below.

In a first embodiment, a decellularized fish skin scaffold material made from minimally processed skin of wild-caught Atlantic cod from Iceland is provided as a skin substitute. For simplicity, in the following subsections, unless otherwise stated, this scaffold material, which is made from minimally processed skin of wild-caught Atlantic cod from Iceland will be termed a “scaffold” or “scaffold material”, which is provided as an embodiment of a skin substitute.

The following is a description of methods for adding one or more color agents to scaffolds and to increase fastness of the color agent(s).

As a preferred embodiment, the general process used to add the color agents of methylene blue (MB) and/or gentian violet (GV) to the scaffold.

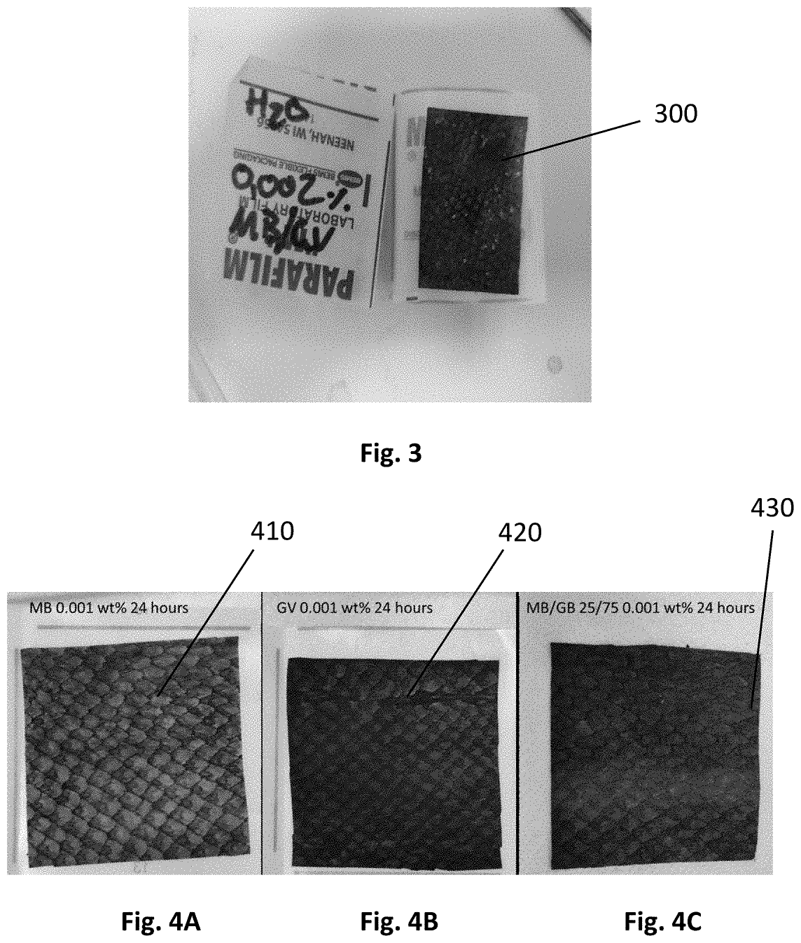

In an exemplary procedure, 100 mL of a dye solution is used (either based on deionized water or phosphate-buffered saline (hereafter abbreviated “PBS”) containing 0.001 wt % of each colorant (MB and GV) (in total 0.002 wt % total or 20 mg/L when the two dyes (MB and GV) were used). A piece of freeze-dried scaffold, approximately 4×4 cm in dimensions and weighing 0.25-0.30 g, is added to the solution and left for 3 hours. The Kroma scaffold is then removed from the solution and washed with tap water before being rinsed with deionized water and frozen. The resulting dyed scaffold 300 is shown in FIG. 3.

When used as described above with MB and GV, the combined amount of both dyes in the scaffold is approximately 1 mg/g. The exact same method can be used with any combination of the four dyes mentioned above (methylene blue (MB), gentian violet (GV), brilliant blue FCF (BB-FCF), and indigo carmine (IC)) or one of these four dyes, either at the same total concentration (0.002 wt %) or per dye (0.001 wt %) concentration in water or PBS solution. Slight variations in the total concentration or the volume of the dye solution would yield the same total concentration in the scaffolds for any single dye or other combination of the dyes listed above. A UV-VIS spectrophotometer can (and was used by the inventors) to measure the absorbance, and by extent the concentration, of the coloring solutions before and after the dyeing process with any single dye, or any combination of dyes, to determine the affinity for adsorption to the scaffold.

FIGS. 4A, 4B, and 4C show the resulting scaffold materials dyed with a 100 mL of a dye solution (either based on deionized water or phosphate-buffered saline (hereafter abbreviated “PBS”) containing 0.001 wt % of MB in the scaffold material 410 of FIG. 4A, which was left for 24 hours; containing 0.001 wt % of GV in the scaffold material 420 of FIG. 4B, which was left for 24 hours; and containing total amount of 0.001 wt % of a combination ration of 25/75 MB/GV scaffold material 430 of FIG. 4C, which was left for 24 hours.

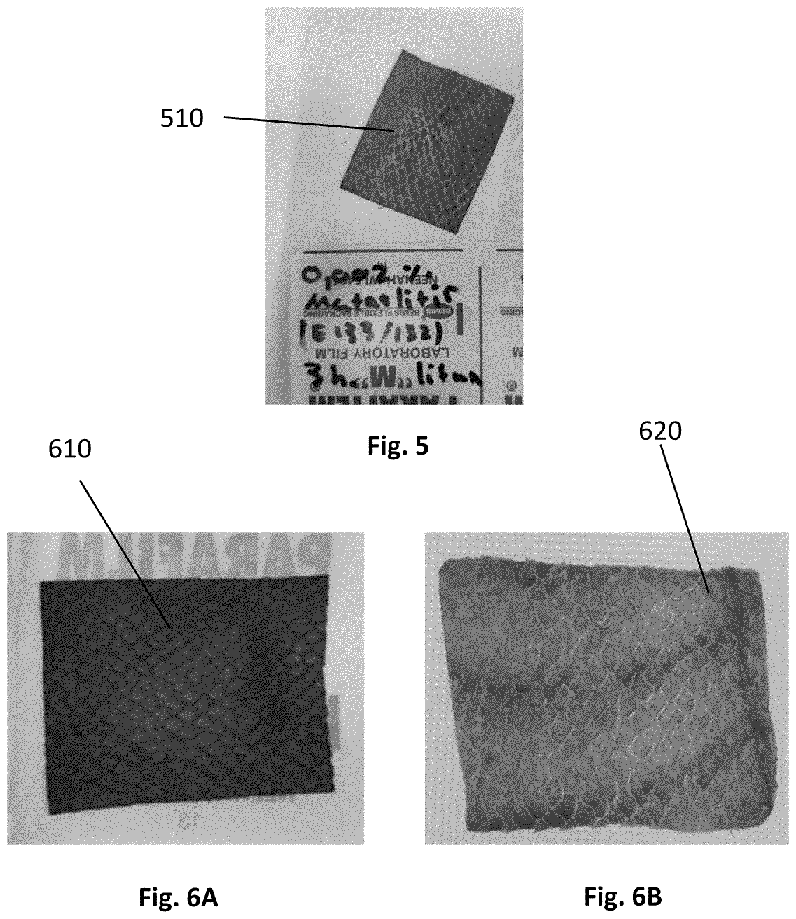

In other embodiments, other additional combination of dyes were used, including: (1) BB-FCF and IC applied together, using the same experimental setup as above (when MB and GV are applied); (2) BB-FCF and/or IC applied before or after MB and/or GV for increased longevity at in vivo conditions; and (3) BB-FCF and/or IC in combination with MB and/or GV. In the above combinations, the solvent could be either water or PBS. Other embodiments include other solvent mixtures discussed herein in later sections. FIG. 5 shows the resulting scaffold 510 dyed with BB-FCF and IC applied in a similar weight to the above-noted examples with colorants MB and GV as above, at 0.001 wt % of each colorant BB-FCF and IC, with a total concentration 0.002 wt %, left for 3 hours.

Alternative methods using mordants

In other embodiments, difference methods or color fasteners are used to increase the fastness of a color agent or combination of color agents added to the scaffold material.

Mordants, or dye fasteners or fixatives, are a group of compounds used in biological staining and in the textile industry, which are largely comprised of metals with a valency of two (salts). Compounds such as tannic acid or cream of tartar (potassium salt of tartaric acid) are often used in the same purpose, although they generally not considered to be true mordants.

The choice of mordant often depends on the dye being used, for example some zinc salts may be used with MB and iodine (KI+12) for GV. Iodine is also used in gram stains as a mordant, although it is rather considered a trapping agent than a true mordant.

One definition of a mordant is a polyvalent metal ion which forms coordination complexes with certain dyes, although this definition is not necessarily limiting in this disclosure to the term “mordant”, as reflected above, some compositions are generally considered to be mordants by those of skill in the art (e.g., tannic acid, cream of tartar, iodine) despite not falling within this definition.

Mordants can in general be applied in the coloring process in three ways: (1) Pre-mordanting (onchrome), wherein the substrate is treated with the mordant and then the dye; (2) Meta-mordanting (metachrome), wherein the mordant is present in the coloring solution from the beginning (This process is simpler than pre-/post-mordanting but is only applicable with a small number of dyes); and (3) Post-mordanting (afterchrome), wherein the substrate is first treated with the dye and then with the mordant.

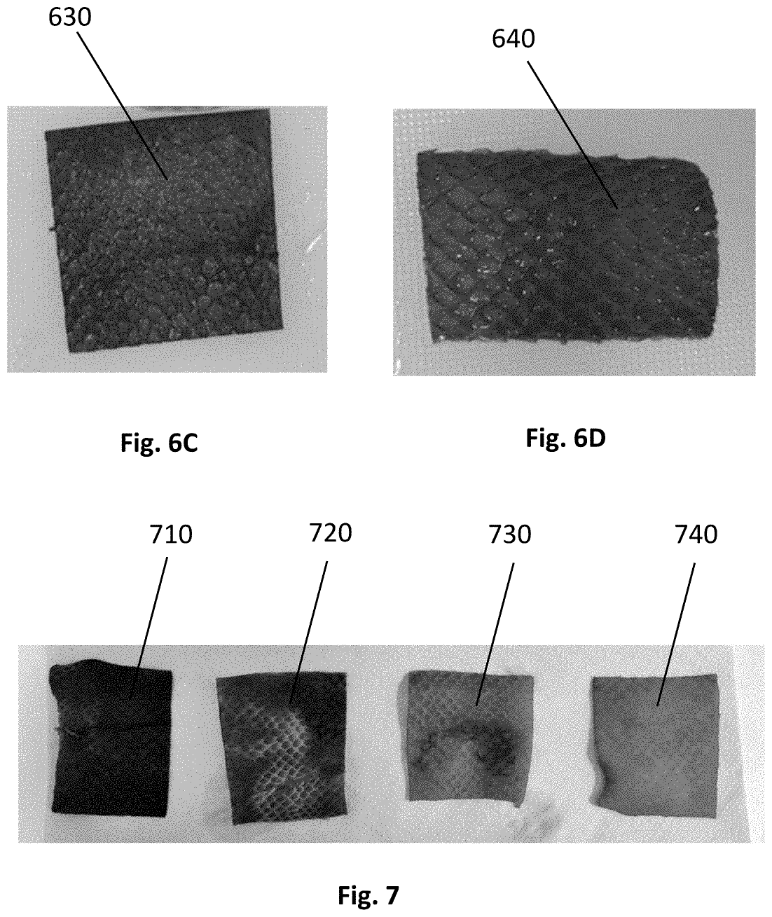

FIG. 6A and 6B show mordanted scaffold materials, with FIG. 6A showing a post-mordanted sample of Kroma scaffold material 610 having been dyed with a combination of MB/GV at a concentration of 0.002 wt % using alum, and FIG. 6B showing a pre-mordanted sample of a scaffold material 620 having been dyed with a combination of MB/GV at a concentration of 0.002 wt % using alum. Alum, also known as aluminum sulphate, is one of the most used mordants for textiles as it provides good fastness for a variety of dyes as well as increasing the brightness and saturation of the color. However, it is by no means the only possible mordant that is usable with scaffolds. In other embodiments, possible mordants/salts include, but are not limited, to NaCl, MgCl2, MgSO4, CaCO3, CaCl2, KCl, ZnCl2, some other Zn salt or even using KI/12.

In other embodiments, as shown in FIGS. 6C and 6D, variations of meta-mordanting, a scaffold material 630 dyed with a combination of MB/GV at a total concentration of 0.002 wt % in FIG. 6C, wherein 0.5 grams of CaCl2 was added. And in FIG. 6D, a scaffold material 640 dyed with a combination of MB/GV at a total concentration of 0.002 wt %, wherein 0.5 grams of NaCl was added.

In embodiments, any, or a combination of two or more, of these metal salts/mordants are used with any dye or any combination of dyes described above, or any other dye combination and coloring technique.

In different embodiments, all three mordanting methods are applied for approx. 2 hours at approximately 90° C. In cases where that is not possible, the scaffold substrate can be kept in solution for up to or over 48 hours at room temperature. In our case, the inventors experimental results show that heating the scaffold in sodium chloride solution at approximately 80° C. for 2 hours may lead to a substantial breakdown of the collagen into a more gel-like form that most likely is due to partial decomposition of the collagen to gelatine. Therefore the “cold method” or a hybrid method, e.g. approx. 37° C. for 12 hours, is preferred.

Meta-mordanting may in general be considered the most restricting method. This is due to a variety of factors, including the solubility of the dye-mordant complex that forms during the process (known as dye-lake). The solubility of the complex is often lower than of the mordant and the dye separately causing it to precipitate, which limits the mordant-dye combinations that can be applied. Additionally, when using meta-mordanting the time in solution is dependent on the mordanting time, which is approximately two days at room temperature. Therefore, the dyeing process may need to be longer or at a higher temperature. As the quantity of adsorbed color has been shown to be directly linked to time in solution, and is most likely also correlated to temperature.

Preferred embodiments include pre- or post-mordanting where post-mordanting is potentially more favourable for our application as it can be used without changing the current coloring and quantification methods given the scaffold does not lose much color during the mordanting process. The pre-mordanting process might alter the adsorption rate of the dye as the dye-lake forms directly on the surface of the scaffold during adsorption. Additionally, the mordant could “leak into” the dye solution potentially causing precipitation or change in the intensity of absorbance of the dye molecules. In either case this would cause issues when measuring the amount of dye adsorbed by the scaffold.

As stated earlier, alum is a preferred mordant, but in other embodiments, other mordants may be used, including, but not limited to, metal salts, such as salts of sodium, magnesium, potassium and iron.

pH gradient dyeing or “through dyeing”

In other embodiments, the process of “through dyeing” was used in the dyeing processing of scaffolds. The process involves a gradual change in the pH of the dye-solution during the dyeing process, from slightly basic, pH 9-10, to slightly acidic, pH 3-4. This can be accomplished using a variety of either weak acids/bases (e.g. acidic acid/sodium bicarbonate), diluted solution of strong acids/bases (e.g. HCl/NaOH) or using extremely small amounts of concentrated strong acids/bases or a combination of these methods. This process can be used instead of or along with any of the dyeing methods described herein. The effect of though dyeing can likely be attributed to the limited stability of the tertiary structure of proteins (in this case collagen) over the range of the pH scale. When exposed to a pH that the protein has not evolved to handle, it will deform by opening of the protein structure that exposes possible binding sites for the dye.

In FIG. 7 various resulting scaffold materials are shown in which a dyeing process is accompanied with a pH grading. First, a scaffold material 710 is shown after dyeing with a combination of MB/GV at a total concentration of 0.002 wt %, when applying a pH gradient. A scaffold material 720 is shown after dyeing with MB at a total concentration of 0.002 wt %, when applying a pH gradient. A scaffold material 730 is shown after dyeing with a combination of MB/BB-FCF at a total concentration of 0.002 wt %, when applying a pH gradient. And scaffold material 740 is shown after dyeing with BB-FCF at a total concentration of 0.002 wt %, when applying a pH gradient.

Unconventional dyeing or fastening methods

In other embodiments, unconventional dyeing or color-fastening methods are used. Following are descriptions of several of the unconventional dyeing methods that have been used in other embodiments. In these embodiments, the effectiveness of the color absorption or the degree of fastening was determined primarily by a visual inspection.

In some embodiments, an alternative solvent was used in the dyeing process. The above-described dyeing methods were performed using a water-based/aqueous solvent for the dyes. However, the four above-noted dyes (MB, GV, BB-FCF, and IC) are soluble in a variety of solvents, like ethanol, and slightly lipophilic in addition to being soluble in water.

In some embodiments, MB and/or GV are dissolved in ethanol and the freeze-dried scaffold is dyed in the ethanolic solution. This resulted in a much lighter coloring when compared to a similar aqueous method, and even when a more concentrated dye solution and a longer dye time was applied. Although a lighter overall coloring was visually seen in these embodiments, these embodiments may still be considered effective and even preferable, as the color fastness may be improved and the potential for a permanent tattooing of the wound may be less likely to occur, while still providing an effectively colored skin substitute.

In other embodiments, dyes are dissolved in oleic acid, but their solubility was lower in these embodiments. However, using a 70/30 oleic acid/ethanol mixture yielded higher solubility. It has been found that the resulting color of the scaffolds was overall darker than for ethanol and oleic acid for the same time and concentration of dye.

In other embodiments, vegetable oil was also used. And fish-oil/cod liver oil can also be used. Using an oil/organic solvent-based dye solution can be done with any combination of the dyes and could also be done on pre-mordanted scaffolds with a variation of oils, fatty acids, their salts and solvents.

In other embodiments, coating treatments were performed after dyeing. In these embodiments, those of most interest are oil and sugar-based coats.

In one embodiment, a mixture of triglycerides, monoglycerides and free fatty acids derived from fish oil, were used for coating by spraying a thin film on a scaffold after dyeing. The sample gained some resistance to the in vitro decoloring and breakdown experiments compared to a similar sample without coating. Additionally, this could also be done using suitable fatty acid alkyl esters.

In other embodiments, sugar-based coats are made with a variety of sugars, either simple sugars (monosaccharides) like ribose, fructose or dextrose or double sugars (disaccharides) like sucrose or maltose. The chosen sugar is dissolved in a water solution, and the scaffold is submerged before being freeze-dried again. Non-reducing sugars may be dissolved in the coloring solution. Sugars may increase the stability of the collagen itself, by introducing additional crosslinking. In addition, sugars contain a high amount of —OH groups which may promote additional linkage to the dye molecules by hydrogen bonds or dipole forces. Additionally, nitrogen containing sugars like N-acetylglucosamine might form covalent bonds with free amino/acid ends of collagen and certain dyes.

Although almost all the possible methods and embodiments described herein above may be used together, using multiple components of each category, for example two or more mordants, to increase the vibrancy, fastness, etc., of the color this may also increase the complexity, possible side effects, and overall cost of producing the dyed scaffold.

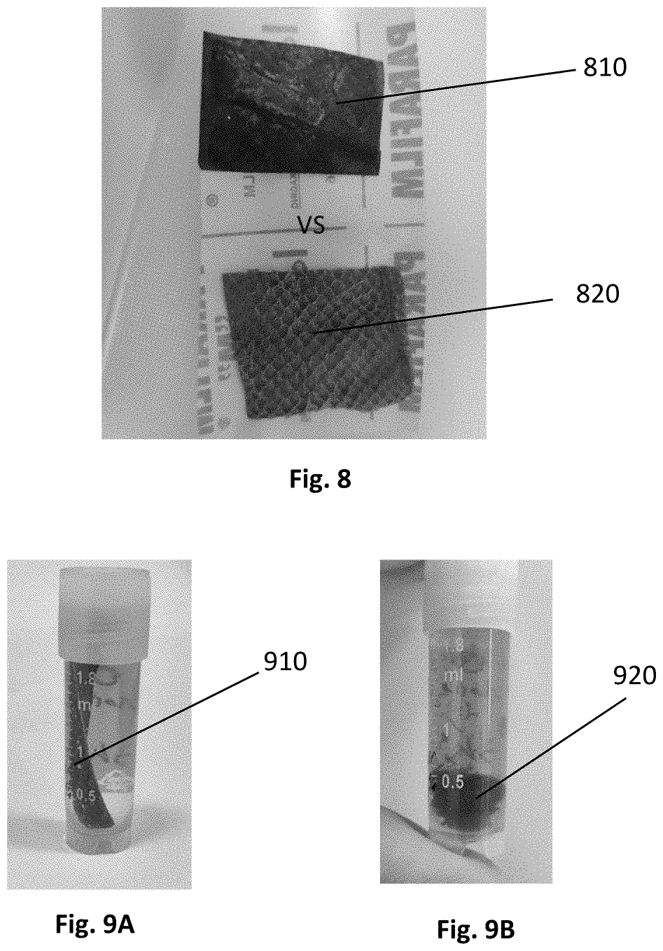

Therefore, a preferred method and embodiment that yields a suitable prototype is similar to the “basic” dyeing process. The most notable issue identified is the longevity of the dye in in vivo condition (e.g., in mice). A potential improvement is a mordanting step, pH gradient, or a combination of the two could possibly be used to increase the binding of the dye molecules within the collagen matrix of the scaffold. As shown in FIG. 8, two preferred embodiments of dyed scaffolds are shown in comparison. Scaffold 810 is dyed using a combination of MB/GV at a total concentration of 0.002 wt %, and scaffold 820 is dyed using a combination of MB/GV at a total concentration of 0.002 wt % and a pre-mordanted dye.

Collagenase catalyzed degradation of scaffolds

When a scaffold is used as a biological dressing the body breaks the large scaffold down into a “pool” of microscopic pieces, which help to rebuild and grow the affected area. In validation experiments, a solution of collagenase in PBS was chosen to mimic this degradation process of the scaffold and its influences on dye(s) in scaffold samples. In nature and people, the primary function of collagenase is to break down collagen to the peptide level, which happens, for example, in damaged tissues within the skin, and which helps the body generate new healthy tissue.

For this a stock solution of 0.50 mg/mL collagenase in PBS was prepared. In the first experiment that stock solution was then diluted to 10 or 100 μg/mL. In total 9 solutions were made using either PBS or “human plasma like solution” as the bulk of the solution, 10 ml, pieces of scaffolds were then put in solution and kept at room temperature for an extended period.

It has been found by the inventors that the un-dyed scaffold starts to break down in the collagenase PBS solution. For the plasma-like medium the solution seemed to inhibit the collagenase as was seen by the comparative level of breakdown of the a scaffold after approximately 3 days, even in the case that the concentration of collagenase was 10 times higher in the plasma solution. Not much change was seen in a scaffold dyed with a combination of MB and GV at a concentration of 0.002% over this time.

To break down the MB/GV-dyed, a considerably higher concentration of collagenase was applied. For this the original 0.5 mg/mL PBS solution was used. After a piece of un-dyed scaffold had been tested for comparison of time and level of break down a series of variations were tested. It was found that the total breakdown into microscopic particles only took approximately 24 hours. In turning to dyed samples, as shown in FIG. 9A, a first scaffold sample 910 dyed in a 0.001 wt % solution of MB/GV was tested. In a 0.5 mg/mL collagenase solution, the total breakdown of that sample took approximately 2 days. As can be seen in FIG. 9B, some of the color has bled into the solution, however most of the dye is still bound to the small collagen particles 920.

Next five of the “prototypes” described in previous sections were tested in the same way. The prototypes tested were scaffold materials dyed in 1) 0.002 wt % MB/GV in water, 2) 0.002 wt % MB/GV in PBS, 3) 0.002 wt % MB/BB FCF in PBS, 4) 0.001 wt % IC in water, and 5) 0.002 wt % MB/GV in water coated with a mixture of triglycerides, monoglycerides and free fatty acids derived from fish oil. Over the next 4 days the samples were checked, in all except one case (sample 4: 0.001 wt % IC in water) the total degradation took 4 days.

In these experiments, it was shown that the dyed scaffolds (1-5, above) broke down into microscopic pieces and in all cases a good part of the dye remained on those pieces. This suggests that the dyes are securely bound to the collagen/peptides of the scaffold and not only to the surface. As stated above, in all except one case (i.e., sample 4: 0.001 wt % IC in water) the degradation took approximately 4 days, and not much difference was observed between samples dyed with MB/GV. Samples 3 and 4 were slightly different, sample 4, dyed with IC was almost completely broken down in just under 24 hours, leaving only a few small pieces. Sample 3 was more stable compared to sample 4 but broke down faster and into smaller pieces than the other three samples.

In the above embodiments, the coloring of the scaffolds entailed generally a combination of Methylene Blue (MB) and Gentian Violet (GV) in equal weight ratios. However, this is not necessarily the case. The dyeing amounts may be tuned to yield scaffolds with a specific amount of colorant, with the aim to have the amount of colorant to be below, and in some embodiments considerably below the maximum allowed amount of MB issued by the FDA for this kind of product. The combined amount of both dyes MB/GV in the scaffold is approximately 1 mg/g, but a maximum allowed amount may be 2 mg/g, 3 mg/g, 4, 5 mg/g, 6 mg/g, 7 mg/g, 8 mg/g, mg/g, 9 mg/g, or even 10 mg/g in some embodiments.

In some embodiments, the step of adding the colorant uses 100 mL of a dye solution (either based on deionized water or PBS) containing 0.001 wt % of each colorant (in total 0.002 wt % total or 20 mg/L). However, this amount of colorant within the dye solution may be increased or decreased to meet the needs of the skin substitute to which the colorant is to be applied. For example, dye solution may have an amount of 1.0 to 10.0 wt % colorant or color agent (either based on deionized water, PBS, or some other dye solvent), 1.0 to 20.0 wt % colorant or color agent (either based on deionized water, PBS, or some other dye solvent), 1.0 to 0.01 wt % colorant or color agent (either based on deionized water, PBS, or some other dye solvent), 0.01 to 0.001 wt % colorant or color agent (either based on deionized water, PBS, or some other dye solvent), 0.05 to 0.002 wt % colorant (either based on deionized water, PBS, or some other dye solvent), 0.01 to 0.0002 wt % colorant (either based on deionized water, PBS, or some other dye solvent), or 0.01 to 0.0002 wt % colorant or color agent (either based on deionized water, PBS, or some other dye solvent), depending on the colorant, the skin substitute to which the colorant or color agent is to be added.

A piece of scaffold, approximately 4×4 cm in dimensions and weighing 0.25-0.30 g, may be added to the solution and left for 3 hours. But as described above, the size of the scaffold material and the amount of time the scaffold material is left in the dyeing solution may be varied. Further, the size of the scaffold material can of course be varied, and requisite adjustments may be made to the volume and concentrations of the dye solution. The scaffold is then removed from the solution and washed with tap water before being rinsed with deionized water and frozen, freeze-dried, or lyophilized.

It has been found in some embodiments, that the relative amounts of MB and GV adsorbed by the scaffold changes when PBS is used instead of deionized water as the base of the solution, changing from approximately 60% GV and 40% MB by weight, for water solution, to approximately 40% GV and 60% MB by weight for PBS. However, the total amount of adsorbed colorants is more or less the same. Further, varying MB/GV ratios may be used when MB and GV are used in combination, including MB ratio of 95/5, 90/10, 85/15, 80/20, 75/25, 70/30, 65/35, 60/40, 55/45, 50/50, 45/55, 40/60, 35/65, 30/70, 25/75, 20/80, 15/85, 10/90, and 5/95. The ratio of MB/GV may range from 10 to 50% MB, 10 to 60% MB, 10 to 70% MB, 10 to 80% MB, and 10 to 90% MB, with the remaining corresponding percentage (90 to 50%, 90 to 40%, 90 to 30%, 90 to 20%, and 90 to 10%). In a preferred embodiment, the MB/GV ratio is 50/50. In another preferred embodiment, the MBGV ratio is 75/25. And in another preferred embodiment, the MB/GV ration is 25/75.

In addition to the use of MB and GV as colorants for the skin substitutes, in other embodiments, the use of food colors, or more precisely the active compound (dye/pigment) in food colors is used, either in combination with or to replace MB and GV.

In one embodiment, a fat-soluble and one water-soluble food color is used. The dye in the fat-soluble food color was E133, or Brilliant Blue FCF (BB-FCF), which is a water-soluble molecule with a very similar molecular structure as GV. The dye was approximately 40 wt % of the food color, but the rest of the additives were for “fat-solubility”. The dye in the water-soluble color was E132, or indigo carmine (IC), which was approximately 85 wt % of the food color.

It has been found by the inventors that the food colors alone may be removable from the scaffold material, both by an enzymatic breakdown using collagenase and by leaving it in a solution of approximately 1M sodium bicarbonate.

Due to the bind mechanism of the above-noted coloring agents, which have been determined to be bound to collagen/peptides of the scaffold materials, and not only to the surface, through similar bonding mechanisms with similar or appropriate coloring agents on other collagen or peptide-based skin substitutes can be performed.

Scaffold material in accordance with this invention may be obtained from intact fish skin or any species of fish, including bony or cartilaginous fish, can be used as the source of the fish skin. For example, the source can be round fish like cod, haddock and catfish; flatfish, like halibut, plaice and sole; salmonids like salmon and trout; scombridaes like tuna; or small fish like herring, anchovies, mackerel and sardines. Further, other collagen, peptide, or other protein-containing skin substitutes, whether of biological skin substitutes, synthetic skin substitutes, or hybrid skin substitutes may be similar colored with appropriate combinations of dyes, pigments, and/or other coloring agents.

Testing on Mice and Patients, and Results

Mice

Embodiments of a decellularized fish skin scaffold material made from minimally processed skin of wild-caught Atlantic cod from Iceland were provide as a skin substitute. Again, in the following subsections, unless otherwise stated, a “fish skin” used as a scaffold material, which is made from minimally processed skin of wild-caught Atlantic cod from Iceland will be termed a “fish skin” as a “scaffold” or “scaffold material”, which is provided as an embodiment of a skin substitute.

A total of 52 mice were tested using embodiments of colored scaffold materials as a skin substitute.

In a first pilot, Pilot 1, 4 mice were treated.

Pilot 1 included the following:
1) a coloring agent of 0.005 wt % MB+0.005 wt % GV, freshly decellularized fish skin stained in water for 3 hours before lyophilization; and
2) a coloring agent of 0.010 wt % MB+0.010 wt % GV, freshly decellularized fish skin stained in water for 3 hours before lyophilization.

In a second pilot, Pilot 2, 16 mice were treated.

Pilot 2 included the following:
1) a coloring agent of 0.001 wt % MB+0.001 wt % GV, freshly decellularized fish skin stained in water for 24 hours before being dipped into cryo sugar solution and lyophilized;
2) a coloring agent of 0.001 wt % MB+0.001% GV, lyophilized fish skin stained in water for 3 hours before being dipped into cryo sugar solution and lyophilized;
3) a coloring agent of 0.001 wt % MB+0.001 wt % GV, lyophilized fish skin stained in water for 3 hours before being dipped into mineral oil and lyophilized; and
4) a coloring agent of 0.001 wt % MB+0.001 wt % GV, lyophilized fish skin stained in water for 3 hours before lyophilization.

In a second pilot, Pilot 3, 32 mice were treated.

Pilot 3 included the following:
a coloring agent of 0.001 wt % MB+0.001% GV, lyophilized fish skin stained in PBS for 3 hours before lyophilization.

Results of mouse studies of each of Pilot 1, Pilot 2, and Pilot 3 had the following results. No unexpected inflammation or other adverse events were detected after using the colored fish skin as a scaffold material. The treatment products (scaffold material) degraded in the usual amount of time and wounds healed normally. Significantly, permanent or semi-permanent tattooing of the wound bed was not detected.

Human Patients

Three patients (Patient 1, Patient 2, and Patient 3) were treated with minimally processed skin of wild-caught Atlantic cod from Iceland, which is termed a “fish skin” or a “scaffold” or “scaffold material” in this subsection.

In each of the three patients (Patient 1, Patient 2, and Patient 3), the scaffold material was produced as in Pilot 3 above, with a coloring agent of 0.001 wt % MB+0.001% GV, lyophilized fish skin stained in PBS solution for 3 hours before lyophilization.

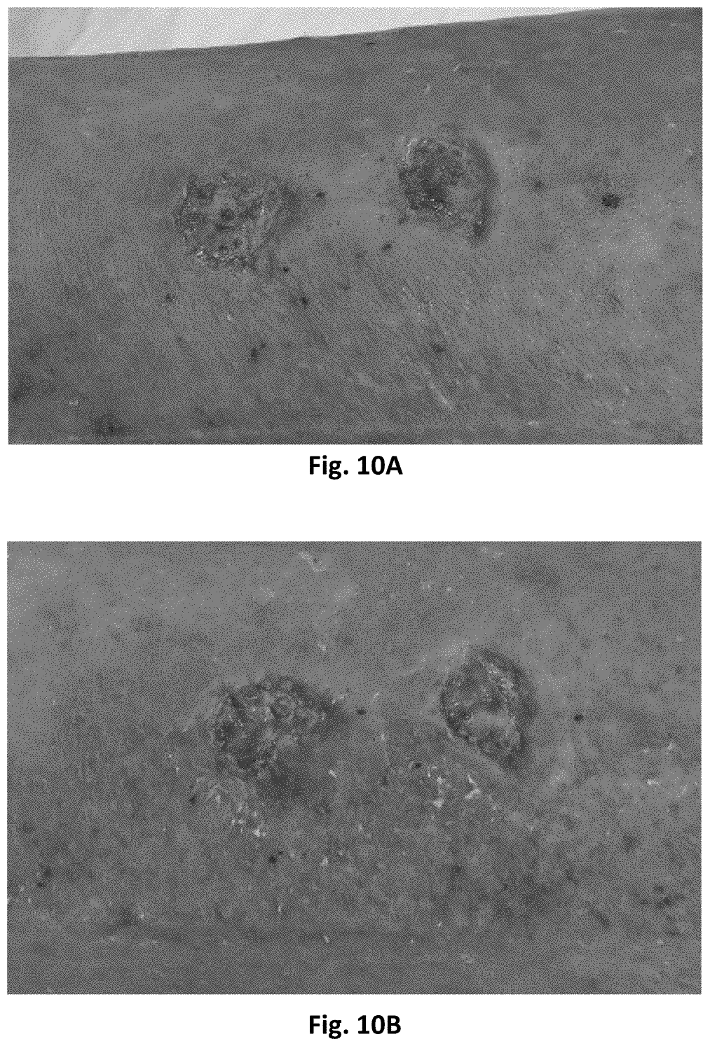

Patient 1 was treated Oct. 12, 2021 with the colored fish skin, with a first photograph as shown in FIG. 10A, and the same wound of Patient 1 was photographed again 7 days later, on Oct. 19, 2021, as shown in FIG. 10B.

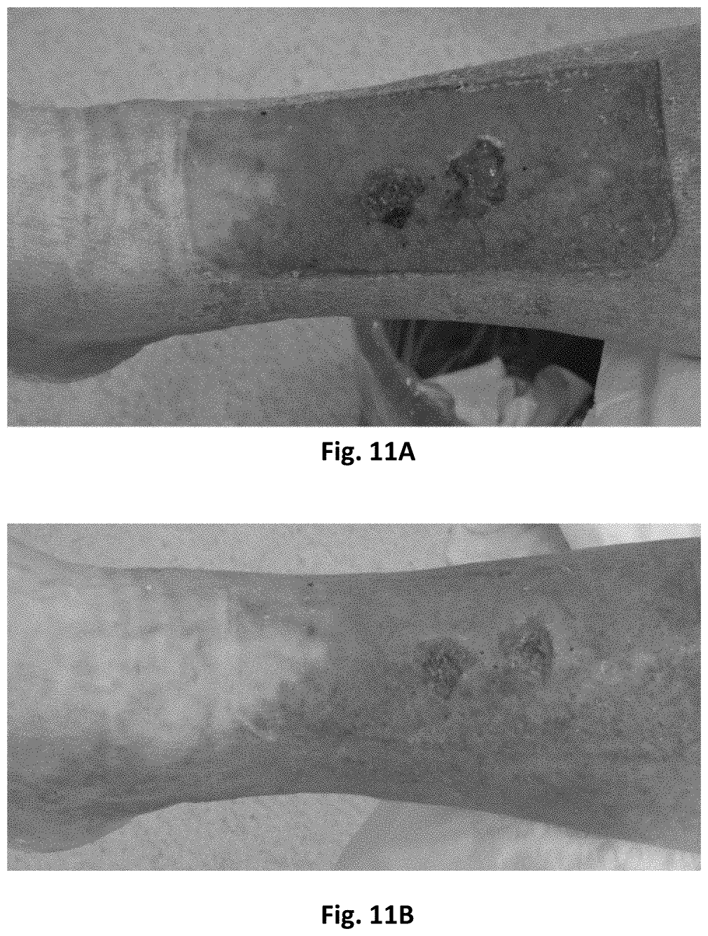

Patient 2 was treated Oct. 25, 2021, with the colored fish skin, with a first photograph as shown in FIG. 11A, and the same wound of Patient 2 was photographed again 7 days later, on Nov. 2, 2021, as shown in FIG. 11B.

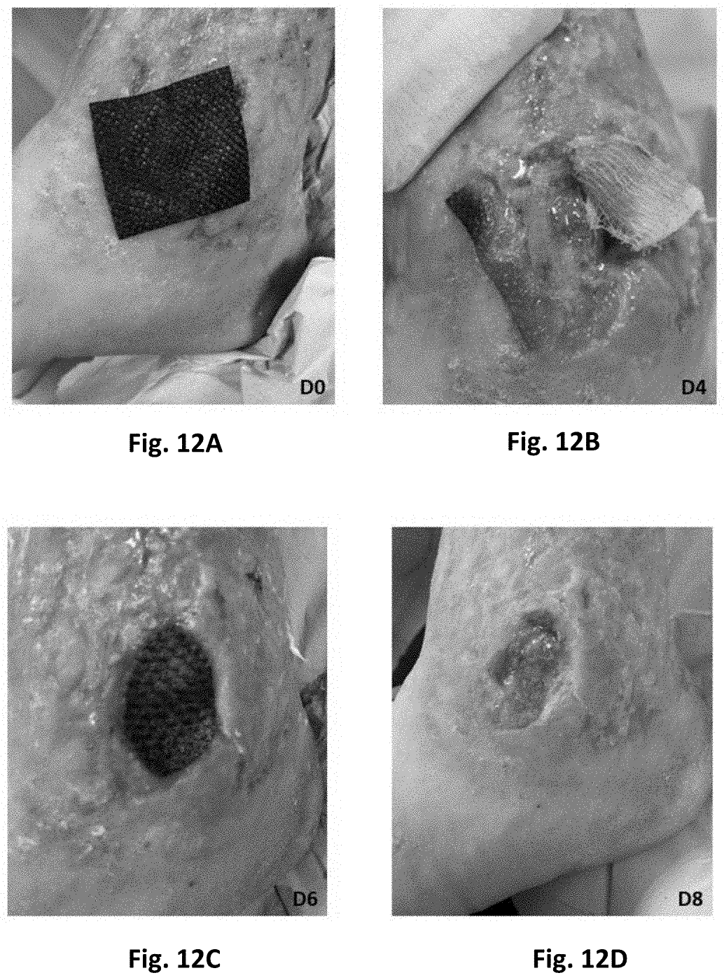

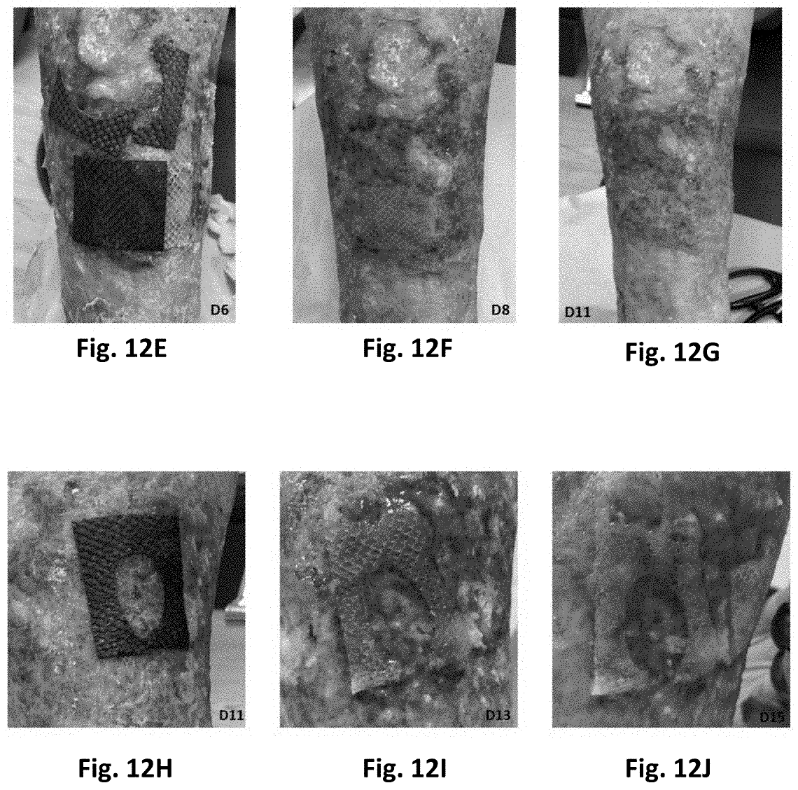

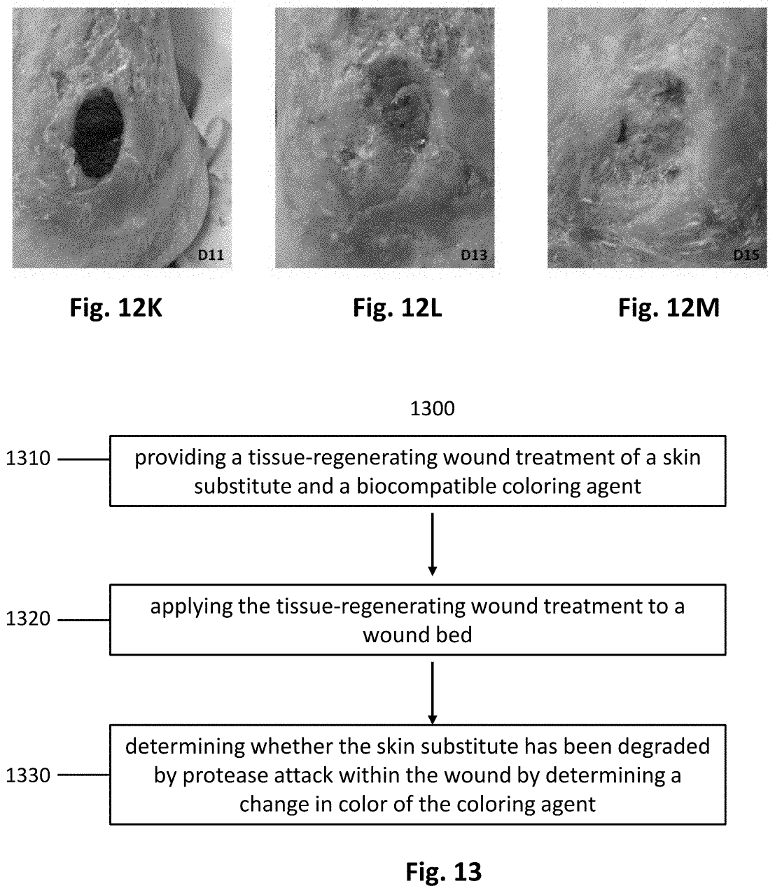

Lastly, various wounds of Patient 3 were treated with the colored fish skin, from Jan. 20, 2022 to Feb. 10, 2022, FIGS. 12A to 12N showing the treated wounds every time a wound dressing was changed. FIG. 12A shows the colored fish skin being applied on Day 0, and FIG. 12B showing the same wound on Day 4. A new colored fish skin is applied as shown on Day 6 in FIG. 12C, and FIG. 12D shows the treated wound two days later on Day 8. With the same Patient 3, a new colored fish skin is applied to a different wound in FIG. 12E, with FIG. 12F showing this same wound after two days, and FIG. 12G showing the same wound after five days. FIG. 12H shows a new colored fish skin applied, and FIG. 12I showing the result two days later, and FIG. 12J showing the result 4 days later. Lastly, FIG. 12K shows a new colored fish skin applied, and FIG. 12L showing healing after two days, and FIG. 12M showing the healing result after 4 days.

In each of Patients 1 to 3 above, no device-related inflammation or any other adverse events was reported after the use of the colored fish skin. The applied treatment can be seen as encouraging healing of these chronic wounds. Further, the applied colored fish skin degraded normally in the wound. Moreover, permanent or semi-permanent tattooing of the wound bed was not detected after day 5.

### FURTHER EXAMPLES

When healing wounds using Kerecis™ fish-skin-derived cellular scaffold product (e.g., as disclosed in U.S. Pat. No. 8,613,957), as noted above, the inventors have found the significant problem that clinicians unwittingly mistake or otherwise struggle to distinguish between the wound healing scaffold and infection. This may be due, at least in part, to the color and/or odor associated with the wound healing scaffold once it starts to break down and integrate into the surrounding tissue; it can sometimes have a similar color as infected tissue (e.g., a purulent infection) and may also be mildly odoriferous, which some may interpret to be a similar odor as infected tissue. Thus, the inventors found that there are problems in the art that could significantly benefit from improved products or improvements to the known products.

One solution would be to false-color the fish-skin-derived cellular scaffold so that it may be more readily identified in the clinic and/or differentiated from surrounding tissue when seated in a wound bed. To that end, the following disclosure provides exemplary data from a series of tests focused on identifying a coloring agent that can remain stable over time and that which may be incorporated into the fish skin scaffold during the processing/manufacturing steps.

A first set of experiments were conducted to determine the stability of various coloring agents within a decellularization solution (termed herein as “Decell solution”) used in the processing/manufacturing of Kerecis™ fish-skin-derived cellular scaffold products, which is made from the minimally processed skin of wild-caught Atlantic cod, as described in U.S. Pat. No. 8,613,957. A Decell solution was prepared in line with EBL M222, and the stability of 6 different coloring agents listed in the following table was tested.

| | Methylene | Sunset | | Gentian | | |
|---|---|---|---|---|---|---|
| Color Type | Blue | Yellow | Rhoamine B | Violet | Allura Red | Fast Green |
| Powder/solution | solution | 90% | 95% | Unknown | 80% | 85% |
| Concentration | 1% w/v | | | | | |
| Solution strength | NA | 1% w/v | 1% w/v | NA | 1% w/v | 1% w/v |
| (water based) | | | | | | |

The Decell solution was prepared in accordance with EBL M222. Each coloring agent was prepared to the solution strength of 1% w/v (e.g., as listed in Table 1). 50 mL of the Decell solution was aliquoted into each of 7 separate plastic tubes securable with a respective lid. A first tube included only the Decell solution, acting as a control. A 0.5 mL aliquot of each of the 6 prepared color solutions was separately added to a corresponding tube containing 50 mL of Decell solution. Any reaction or visible change of the mixture was monitored over time.

The solutions in respective tubes with the respective coloring agents were monitored and documented by photographs at the start of the experiment, after 30 minutes, and after 24 hours of incubation.

It was found that many of the coloring agents were quite bright within the Decell solution at the start. In the first 20 minutes, most of the coloring agents started to fade, with the notable exception of Methylene Blue. This trend continued, and after 24 hours the colored Decell solutions had all turned white or nearly white except the Decell solution with the added Methylene Blue. The Methylene Blue color is therefore considered to be a preferred embodiment of a coloring agent to add during the decellularization stage of manufacturing the Kerecis™ fish-skin-derived cellular scaffold product that is made from the minimally processed skin of wild-caught Atlantic cod, as described in U.S. Pat. No. 8,613,957.

In another embodiment, as shown in FIG. 13 is shown, a method 1300 of treatment of a wound using a tissue-regenerating wound treatment is provided. In step 1310, the tissue-regenerating wound treatment is provided comprising a skin substitute and a coloring agent, the coloring agent being a biocompatible coloring agent that degrades upon protease attack within a treated wound. In step 1320, the tissue-regenerating wound treatment is applied to a wound bed. And in step 1330, it is determined whether the skin substitute has been degraded by protease attack within the wound by determining a change in color of the coloring agent.

In a further exemplary method, the tissue-regenerating wound treatment comprising the skin substitute is in the form of an extracellular matrix, colored with blue color (e.g., MG/GV) is inserted into the wound bed and a secondary wound dressing is applied on top. And in a further exemplary method, upon wound inspection the color of the wound bed is noted. If the color is blue, tissue-regenerating wound treatment is (correctly) considered to be intact and cellular in-growth is (correctly or likely) concluded to be taking place. If the wound treatment is no longer blue, it has become slough and needs to be washed away and a new material applied to the wound bed.

The coloring material used needs to be biocompatible and degrade upon protease attack the matrix itself. It may also not be permanent and leave a permanent color or “tattoo effect” in the wound after healing has occurred.

Additional Testing

First color tests were performed on decellularized fish skin. Tests were performed on fish-skin based wound products to see how the material corresponds to different dye chemicals. The aim was to see how the fibrous collagen material will react with different dyers and if it reacts differently wet or dry.

Test Scheme

Glass bowls, pinsets and closed plastic containers were used for the experiment. These tests were to answer the questions on how the collagen material reacts with different dye types, whether the oil based or water based react better with the protein, whether it holds through washing and at what point in the manufacturing it is best to dye the decellularized fish skin scaffold. The difference dyes/coloring agents/pigments/color additives tested included Woad Powder (HUE-3023); Color additive D&C Green #5 Powder AN0725; Color Additive Ultra Marine Blue H9-03R1; Color additive Liquid FD&C blue #1; Color additive Liquid D&C green #5; Color additive Liquid D&C green #6 oil AM4299; Green Concentrated Food Coloring; and Gamier natural Color, mahogany brown.

Coloring before lyophilization

The first step is to color before lyophilization of the decellularized fish skin. This is done to see how the material reacts with the colors when wet and how the color agent will react in the washing and lyophilization. The test was executed after the de-cellularization step in manufacturing of decellularized fish skin wound product.

The decellularized fish skin scaffolds were kept for 60 minutes in the dye chemical and then washed in a continues running water for 2 hours.

Coloring after lyophilization

The second step of this test is to color the material post lyophilization. This was to see if there is any difference in the scaffold's reactions with the color post lyophilization and whether structure is more open to the dye chemicals. The sheets are then to be lyophilized again.

Test Procedure

1 skin from decellularized fish skin was taken and cut in small pieces. The pieces were respectively put in coloring agents, some in undiluted liquid agent, some in mixture of coloring powder and water/oil or a mixture of coloring agent and hair color developer. The pieces were left for 2 hours, after which the pieces were washed thoroughly and inspected, and pictures were taken. What appeared to be promising pieces, were soaked in water in closed containers and agitated till the next morning. This was to see if the colors will eventually stop dissolving in the water. All pieces were again inspected and washed again. Soaked in pure water, all solutions were colored after five minutes. The promising pieces were sent to lyophilization (frozen at −80° C.) and lyophilized at freeze-drier.

A better understanding of different embodiments of the disclosure may be had from the following description read with the accompanying drawings in which like reference characters refer to like elements.

While the disclosure is susceptible to various modifications and alternative constructions, certain illustrative embodiments are in the drawings described below. It should be understood, however, there is no intention to limit the disclosure to the specific embodiments disclosed, but on the contrary, the intention covers all modifications, alternative constructions, combinations, and equivalents falling within the spirit and scope of the disclosure.

The references used are provided merely for convenience and hence do not define the sphere of protection or the embodiments.

It will be understood that unless a term is expressly defined in this application to possess a described meaning, there is no intent to limit the meaning of such term, either expressly or indirectly, beyond its plain or ordinary meaning.

Any element in a claim that does not explicitly state “means for” performing a specified function, or “step for” performing a specific function is not to be interpreted as a “means” or “step” clause as specified in 35 U.S.C. § 112.

As used herein, the term “treatment” is intended to be understood by its common dictionary definition. That is, the term “treatment” broadly includes medical care and/or medicaments given to a patient for an illness or injury. As should be appreciated by those having skill in the art, a “treatment” includes the use of a chemical, physical, or biological agent to preserve or give particular properties to something. Thus, a “treatment” may be the medical care provided (i.e., in the form of a method or series of prescribed acts), or it may refer to the medicament used to preserve or give a particular property to something.

As a non-limiting example, the particle form of decellularized fish skin disclosed herein can be referred to as a “treatment”—i.e., a medicament used to preserve and/or stabilize a wound or which can provide any of the other disclosed beneficial effects to a wound site. Similarly, in some instances, a treatment includes use of the disclosed decellularized fish skin in particle form within methods for stabilizing and/or protecting a wound.

The terms “decellularized,” “decellularized fish skin,” “acellular fish skin,” and the like as used herein refer to a fish skin made according to any method and includes any embodiment disclosed in U.S. Pat. No. 8,613,957, titled, “Scaffold Material for Wound Care and/or Other Tissue Healing Applications.” The foregoing is incorporated herein by reference in its entirety. Accordingly, the terms “decellularized,” “decellularized fish skin,” “acellular fish skin,” and the like as used herein include descaled fish skin from which a substantial amount of cellular and nucleic acid content has been removed, leaving a complex three-dimensional interstitial structure of native extracellular matrix material (ECM). In general, the decellularization described above is a gentler form of processing than is otherwise required and/or routinely performed on mammalian tissues, which often utilize harsh chemical treatments and/or storage in chemicals (e.g., antibiotics).

The decellularization methods described in U.S. Pat. No. 8,613,957 result in production of a scaffold material that maintains a three-dimensional structure of natural extracellular matrix components, and this allows, in some instances, a physical medium by which stem cells—and other cells contributing to the wound healing process—may migrate across and/or be supported on to promote wound healing. The native structure of extracellular components, such as collagen, is maintained within the decellularized fish skin scaffolding material in addition to other native components such as Omega3 polyunsaturated fatty acids (PUFAs).

Other scaffold materials that are derived from mammalian skin/membranes, such as placental-based wound treatments, may also be used as a skin substitute.

A skin substitute based on decellularized fish skin may be preferable because disease transmission risk from the Atlantic cod (Gadus morhua)—and many other fish species—to humans is non-existent or at least far less probable. Additionally, decellularized fish skin likely does not contain allergenic components, significantly decreasing the risk of allergic or other immune response. Owing to the decreased risk of disease transmission and allergic response, decellularized fish skin is subjected to gentle processing that retains the biological structure and bioactive compounds of the extracellular matrix. Accordingly, decellularized fish skin is denuded of skin cells during processing, but it maintains the natural three-dimensional structure of extracellular components, which provides a natural scaffold to promote wound healing. In contrast, mammalian scaffold materials lack a three-dimensional structure and have lost other natural extracellular components and fail to promote wound healing in the same manner or to the same degree as decellularized fish skin.

While other forms of collagen-based materials may be used as a biological or synthetic skin substitute, reconstituted collagen materials preferably are not harvested through harsh physical and chemical treatments that fail to maintain their native three-dimensional structure, particularly within the natural context of other natural extracellular components. Similar to the mammalian-derived scaffold material discussed above, a lack of a native structure and/or three-dimensional extracellular matrix environment provided by reconstituted collagen materials may render the skin substitute less effective at promoting wound healing. Of course, costs of production and consistency of the skin substitute, and other factors, must be considered in selecting a skin substitute, such that use of such reconstituted collagen materials may indeed be preferable in some cases or applications.

Additional Considerations Regarding Incidence of Infection

Wound treatments are often of necessity applied in austere environments by non-medically trained personnel at or near the point of injury, for example, in combat situations. The inventors have found a significant need for a broad antimicrobial spectrum antimicrobial activity with the tissue regeneration abilities, bacterial barrier, and analgesic properties of a wound treatment, for example, fish skin grafts. The inventors have found that a wound treatment product that is easy to store and carry and can act as either a definitive or temporary treatment would be particularly helpful, for example, by reducing the need for evacuation of wounded personnel in combat or emergency situations.

As noted above, infection is a major challenge in emergency and combat wound management. It determines the morbidity and mortality of injured people, or service members on the battlefield. For example, infection accounts for one-third of total casualties, prolonged treatments, and an increase risk of amputation. Because of the distinct mechanisms of injury and the austere environment, combat wounds are prone to contamination, making treatment more difficult. An early sign of infection is bacterial imbalance within the wound. Common pathogens found in the wound at an early stage include both gram-positive (G+) and gram-negative (G−) strains. In the event of an infection, an emergence of gram-negative bacteria and multi-drug resistant (MDR) organisms are observed. The inventors have therefore identified a great need for an effective and immediate intervention to lower the risk of infection to benefit soldiers and emergency personnel.

The tissue-regenerating wound treatment of the present disclosure, which in some embodiments may be a blue antimicrobial Fish Skin Graft, provide a novel visual cue of wound healing. The wound treatment of the present disclosure retains the performance benefits of earlier wound treatments, such as graft which accelerates wound healing and provide a biologic covering in burns, acute and chronic wounds. But additionally, the wound treatment of the present disclosure is impregnated with antimicrobial agents, either in the form of antimicrobial coloring agents, such as Methylene Blue (MB) and Gentian Violet (GV), or as a further added active agent. The wound treatment of the present disclosure integrates into the wound bed over time, releasing the antimicrobial agents to prevent onset of infection. The blue color of the skin graft will help reduce unnecessary reapplications, thus minimizing wound exposure, and encouraging wound healing without permanent discoloring of periwound tissue.

Traditional field dressings available in combat or emergency environments may provide immediate cover, may be field deployable in austere environments, may be usable by the patient himself or herself or by a buddy, and often can be used with a saline solution to rinse or dehydrate. However, traditional field dressings do not provide broad antimicrobial coverage, traditional field dressings must be changed daily, and traditional field dressings do not integrated into a wound bed, do not augment wound healing, and do not provide a visual aide of integration for selfcare of care by others.

Antimicrobial silver dressings, which also may be used in combat or emergency environments may provide immediate cover, are field deployable in an austere environment, may be usable by the patient himself or herself or by a buddy, and may provide broad antimicrobial coverage. However, antimicrobial silver dressings cannot be used with saline to rinse or rehydrate, must be changed every 1-3 days, do not integrated into a wound bed, do not augment wound healing, and do not provide a visual aide of integration for selfcare of care by others.

By comparison, the wound treatment of the present disclosure provide immediate cover, are field deployable in an austere environment, are usable by the patient himself or herself or by a buddy, and provide broad antimicrobial coverage. Further, the wound treatment of the present disclosure can be used with saline to rinse or rehydrate, need to be changed only very 5-10 days (based on a color visual aid), and significantly the wound treatment of the present disclosure integrates into a wound bed, augment wound healing, and provide a convenient and effective visual aide of integration for selfcare of care by others.

The wound treatment of the present disclosure is well suited for the combat environment or emergency environments as it considers and addresses the needs of soldiers and medical personnel for the following reasons:
Antimicrobial Activity: methylene Blue MB is a potent antimicrobial dye against G− bacteria. It reduces bacteria burden in wounds and decreases hypergranulation. GV is an antimicrobial dye against G+ bacteria and can impact proinflammatory mediators;
Shelf Life: the wound treatment is stable at RT for 3+ years and is robust against impacts. Stability at prolonged high temperature and humidity will be examined;
Packaging: the wound treatment in preferred embodiments is individually packaged in a vacuum sealed, military grade, foil pouch containing a dry, sterilized sheet of fish skin. The pouches are small, lightweight, and fit easily in a pocket or medical bag (100 cm2 fish skin 2g). The packaging is resistant to humidity and austere environments. The product is easy to transport, store, and is available in multiple sizes;
Ease of Use: the wound treatment requires basic medical supplies and limited medical knowledge to use. The color agent can assist users in distinguishing the wound pus/slough and the integrated fish skin in the wound bed, allowing straightforward follow-up treatments;
Non-staining: the wound treatment uses medical grade color compounds with known coloring and breakdown profiles. No staining has been seen with regular topical use. If any pigment is absorbed, the breakdown profile has been found by the inventors to be 6 to 12 days;
Removable: the wound treatment does not need to be removed from the wound. The skin substitute, such as fish skin, recruits native human cells into its structure where the cells eventually convert the skin substitute, such as fish skin, into new tissue. If required, however, the product can easily be removed by lifting it with tweezers or wiping it off with a moist gauze when the fish skin begins to integrate;
Usage in austere environments: the wound treatment can be used at or close to the point of injury as either definitive wound therapy or temporary antimicrobial cover. The skin substitute, such as fish skin, slowly integrates, which results in fewer frequent dressing changes;
Pain reduction: the wound treatment uniquely provides a skin cover for the wound, creating an inside body environment. The graft, which in the case of fish skin, is rich in fatty acids including Omega3, helps shield the exposed nerve endings, reduces inflammation and positively influences pain via lipid mediators.

A significant objective of this disclosure is to provide to the Department of Defense and Emergency Personnel with an innovative solution for wound management at or near to the point of injury as an FDA cleared antimicrobial skin substitute. The wound treatment of the present disclosure will provide superior healing properties together with potent antimicrobial activity. The wound treatment could be applied as a definitive care for smaller, less serious wounds and a temporary antimicrobial cover for severe injuries that need transfer to higher echelons of care. Additionally, the color agent will help medical care providers distinguish between the integrating skin substitute and pus or wound slough.

The wound treatment of the present disclosure will promote wound healing through a combination of the following approaches: 1. Acting as an extracellular matrix that integrates into the wound, providing structural support for the host cells to heal and regenerate tissue. 2. MB and GV inhibit G+ and G− bacteria along with fungi, thus preventing biofilm formation and reducing the risk of infection. 3. Fewer dressing changes leads to less wound exposure to contaminations and mechanical trauma from repeated dressing removal. 4. The color guides non-medically trained users in optimal dressing and antimicrobial management. 5. Biomolecules naturally present in the skin substitute, for example, fish skin (Omega3 and collagen), or added active agents, reduce pain, inflammation, and bleeding.

The wound treatment of the present disclosure will provide a definitive and temporary treatment for minor and major wounds/burns by preventing infections, providing coverage, and promoting healing.

Preferred embodiments of skin substitutes, such as decellularized, freeze dried fish skin grafts are extremely effective in initiating and facilitating the natural healing process. A skin substitute, and particularly a physical scaffold, and even more preferably, a physical scaffold of the fish skin, allows the cells to infiltrate and provides biomolecules to reduce inflammation and pain. These properties have been demonstrated many times in in vitro, in vivo, and in clinical studies. Additionally, in the further preferred embodiment of fish skins are rich in natural Omega3, which has been shown to act as a barrier to bacterial invasion, anti-viral potential, bacteriostatic and antibacterial effects.

A preferred embodiment of fish skin provides bacterial barrier properties. Perhaps the most compelling evidence for the abilities of unstained, fish skin in reducing wound infections is an independent, 21 patient study conducted at the Curie Institute in Paris, where the infection rate for split thickness donor sites was reduced from 60% to 0% for fish skin treated wounds. Even an uncolored fish skin graft can act as a bacterial barrier against Staphylococcus aureus for up to 48-72 hours at optimum bacterial growth condition. An in vivo study on an infected mouse model demonstrated that the fish skin can act as a bacterial barrier against P. mirabilis, one of the most frequently identified MDR strains in combat-trauma associated infections.

Methylene Blue and Gentian Violet provide even additional antibacterial properties. Advances in wound treatment has resulted in the combination of antibacterial agents such as silver, iodine, polyhexamethylene biguanide (PHMB) with traditional wound dressings. While silver and iodine display a robust effect in antibacterial activity, the extended use of these agents leads to a high level of cytotoxicity for the host cells. MB and GV are cleared by the FDA and may be used topically and have demonstrated superior effects for the management of chronic wounds with local infection.

In vitro data has shown encouraging results for the prototypes of the embodiments of preferred embodiments of the present disclosure. Tests were based on the ASTM E2149 and the Kirby-Bauer Zone of Inhibition assays. Both assays indicated that fish skin grafts impregnated with MB and GV efficiently inhibit both E. coli or Staphylococcus aureus in solution and on agar plates.

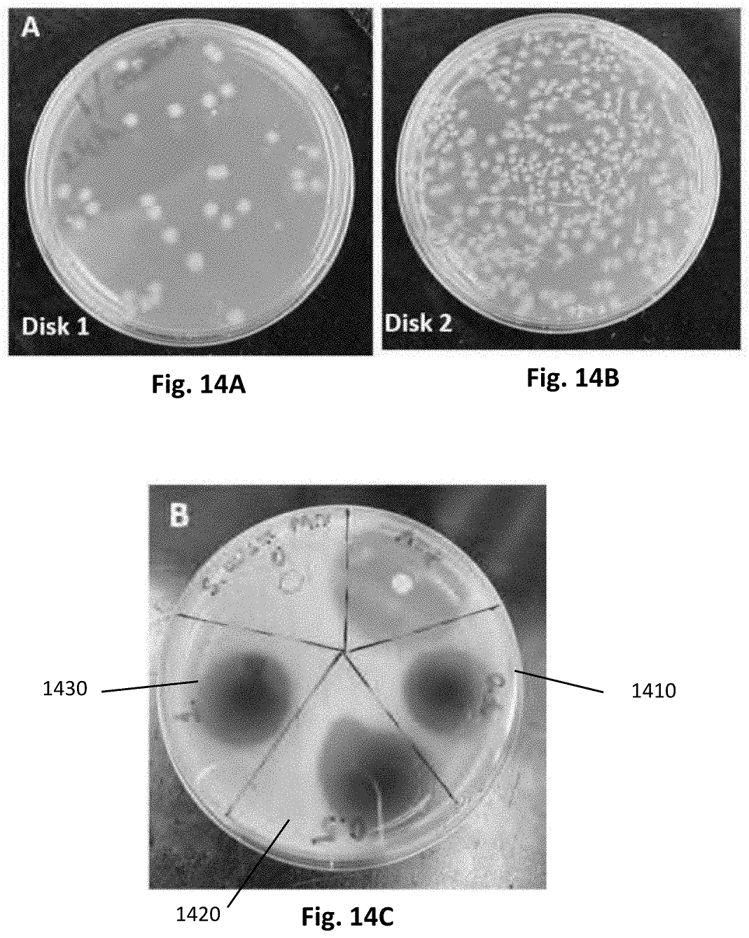

FIGS. 14A and 14B show results from (A) ASTM E2149 on E. coli and FIG. 14C show results (B) Kirby-Bauer Zone of Inhibition assay on Staphylococcus aureus. FIGS. 14A and 14B show results of antibacterial fish skin was placed in an E.coli suspension and was shaken for up to 24 hours, bacterial reduction was clearly observed between the antimicrobial fish skin (Disk 1) (FIG. 14A) and original fish skin (Disk 2) (FIG. 14B). In FIG. 14C, (B) fish skin treated with different concentration of Methylene blue and Gentian Violet ranging from 0.1% w/v (section 1410), 0.5% w/v (section 1420), and 1% w/v (section 1430) showed a clear-inhibition zones on agar plate inoculated with Staphylococcus aureus, no inhibition zone was seen with original fish skin.

Applicant has a wealth of scientific data demonstrating the healing properties of fish skin. This includes two randomized clinical trials on acute wounds, where the fish skin was shown to provide more effective healing as compared mammalian cellular and tissue-based products (CTPs), for example, (Oasis)17 and human amnion/chorion membrane in full healing time, and a clinical donor site study where the use of fish skin halved the healing time for patients. For fish skins as an examplary and preferred embodiment, there have been many independent case series publications with overwhelmingly positive results.

Production of tissue-regenerating wound treatment, and particularly the production of fish skin-based tissue-regenerating wound treatment is very feasible. The added step of impregnating the skin substitute with antibacterial color requires a minimal addition of new equipment. MB and GV are readily available at a pharmaceutical quality grade.

Embodiments of tissue-regenerating wound treatments of the present disclosure, and particularly tissue-regenerating wound treatments comprising fish skin as a skin substitute and having antimicrobial properties provided either by the coloring agent(s) or by further added active agents, may be effective used as a what may be termed a transitory antimicrobial scaffold for the management of wounds including: diabetic foot ulcers, arterial ulcers, pressure ulcers, venous leg ulcers and traumatic ulcers. These wound types combined are thought to be responsible for 54% of lower leg amputations in the U.S., which is an irreversibly debilitating condition. Nearly half of the individuals who have an amputation due to vascular disease will die within five years. This is higher than the five-year mortality rates for breast cancer, colon cancer, and prostate cancer.

The standard of care in the U.S. for treatment of chronic ulcers is as follow: Usual care or standard care for established chronic wounds incorporates common principles, as follows, that apply to managing all wound types; Remove necrotic tissue through debridement (typically sharp debridement); Maintain moisture balance by selecting the proper wound dressing to control exudate; Take measures to prevent or treat wound infections; Correct ischemia in the wound area; For venous leg ulcers, apply some form of compression; For diabetic foot ulcers, apply some form of offloading.

Embodiments of tissue-regenerating wound treatments of the present disclosure, and particularly tissue-regenerating wound treatments comprising fish skin as a skin substitute and having antimicrobial properties provided either by the coloring agent(s) or by further added active agents, may provide a more effective treatment of chronic wounds compared to the SOC established for skin substitutes by being more effective as treatment for chronic wounds by providing a transient scaffolding and resisting bacteria growth within the dressing compared to SOC. For the purpose of this application it is expected that Standard of Care is defined the same way as the Agency for Healthcare Research and Quality (AHRQ) definition. The predicate device decellularized fish skin Wound product has been shown in a randomized clinical trial shown significantly faster healing compared to Standard of Care—Collagen Dressing. The device will provide a more effective treatment compared to the current standard of care as defined by the AHRQ.

The embodiments of tissue-regenerating wound treatments of the present disclosure, and particularly tissue-regenerating wound treatments comprising fish skin as a skin substitute and having antimicrobial properties provided either by the coloring agent(s) or by further added active agents, will achieve the same improvement compared to SOC and at the same time it will furthermore offer resistance to bacterial growth and will have defined characteristics as a transient scaffold.

Although preferred embodiments of tissue-regenerating wound treatments of the present disclosure comprise fish skin as a skin substitute other skin substitutes of course may be used different than fish skin products, or fish skin based wound treatments provided by Kerecis'.

For an expanded comparison the subject device provides a more effective treatment compared to emerging treatments. For this application we include emerging treatments that have been given a Q-code as a skin substitute under the Healthcare Common Procedure Coding System (HCPCS).

As noted above, the group of skin substitutes which can be used as examples of skin substitutes according to the present disclosure is a large and varied. The AHRQ Technology Assessment Program entitled “Skin Substitutes for Treating Chronic Wounds” Technical Brief Project ID WNDT0818, published Feb. 2, 2020, which is incorporated herein by reference, in Table 2, on pages 9-13, identified 76 commercially available products with few studies comparing them internally. Each of these listed skin substitutes may be an embodiment of a skin substitute according to the present disclosure.

For the argument of showing more effective treatments the focus is on the comparison of treatment outcomes, antibacterial properties and transient scaffolding characteristics and their impact on utilization.

The combination of the antimicrobial color to a biodegradable scaffold should at the very least not interfere with the base function of each, and that they could potentially have synergistic additive effects.

A transient scaffolding can be understood to be a tissue scaffold that helps regenerate tissue by supporting cell ingrowth, neovascularization, and the regeneration of extracellular matrix. Current transient scaffolds do not prevent with bacteria growth. Indeed, in some cases the collagen could serve as nutrition for the bacteria. Currently transient scaffold products are not indicated for use in wound care.

An antimicrobial product prevents bacterial colonization of the device, but it does not necessarily help with scaffolding (for silver-based products it might actually be detrimental because of the cytotoxic effects).

The tissue-regenerating wound treatments of the present disclosure, and particularly tissue-regenerating wound treatments comprising fish skin as a skin substitute and having antimicrobial properties provided either by the coloring agent(s) or by further added active agents provide what may be termed “device identification.” When absorbable dressings are applied to a wound it can become hard to identify what is the active but partially absorbed device or what is wound slough that should be removed. This can lead to untimely dressing changes. Currently no absorbable wound product has color identification.

The combined scaffold and antimicrobial color provide synergistic additive benefits.

The tissue-regenerating wound treatments of the present disclosure, and particularly tissue-regenerating wound treatments comprising fish skin as a skin substitute and having antimicrobial properties provided either by the coloring agent(s) or by further added active agents is the first wound care product known to the inventors that provides transient scaffolding and a device identification of an absorbable wound dressing. The combined with antimicrobial protection limits the risk of bacteria causing inflammation or growing into the product, causing accelerated breakdown. Furthermore, easy identification of the device allows for more accurate dressing changes.

Transitory scaffolding may be compared to other skin substitutes. A transient scaffolding supports cell ingrowth, neovascularization, and the regeneration of extracellular matrix. As the field of tissue engineering continues to evolve, the criteria for an ideal skin graft have shifted toward the material that supports cells integration and tissue growth. Those criteria include that the scaffold should satisfy one or more of the following, and preferably all of the following: allow and promote cell ingrowth; allow a uniform spatial distribution of cells; support the regeneration of extracellular matrix; support neovascularization; not trigger foreign body-type reaction; quickly integrate to the wound; be mechanically strong and stable.

The inventors have shown that the fish skin graft technology as described in the present disclosure can provide transient scaffolding function. Furthermore, the evidence shows that the scaffolding results on cell ingrowth, neovascularization, and the regeneration of extracellular matrix are more effective than other known devices, such as an absorbable collagen device, for example, Primatrix.

Based on these results there is evidence that the tissue-regenerating wound treatments of the present disclosure, and particularly tissue-regenerating wound treatments comprising fish skin as a skin substitute and having antimicrobial properties provided either by the coloring agent(s) or by further added active agents provides for more effective treatment than standard of care by acting as a transient scaffold.

The addition of antimicrobial agents on the original fish skin provides antimicrobial protection for the device. And it has found by the inventors that the addition of appropriate color agents does not interfere with the fundamental scaffolding effects of the fish skin. MB and GV are organic dyes that can be used to reduce microbes in clinical setting with minimal toxicity to humans. MB and GV can been used topically for prompt management of localized bacterial burden in wounds. The concentration of MB and GV in preferred embodiments is controlled at equal or less than 0.00025 g/g (0.01%), lower than the concentration in Hydrofera Blue Ready (equal or less than 0.0035 g/g of each color) and significantly under the concentration of commercialized topical agents 1% MB and GV. Of course Hydrofera Blue Ready may be used as nother embodiment of a coloring agent. GV and MB can be used in conjunction with enzymatic debriding agents, growth factors and hydrogels without inhibiting actions of the companion products.

MB and GV as used in tissue-regenerating wound treatments of the present disclosure, and particularly tissue-regenerating wound treatments comprising fish skin have been found by the inventors to not compromise the fish skin's scaffolding effects. Adding MB and GV to the fish skin may be performed at the final stage in the manufacturing process before sterilization. This step will not alter the design, material, function, packaging and sterilization of the original fish skin.

A recent study (Stone II, International Journal of Molecular Sciences, 2021) was conducted to compare the fish skin graft to fetal bovine dermis (Primatrix) in treating deep partial thickness (DPT) burn wounds on a preclinical porcine model. The goal of this study was to determine how well the fish skin graft works on DPT burn wounds, how it integrates and if it improves long term healing. In the conditions of the study, fish skin graft were found to integrate faster into the wound bed than fetal bovine dermis. The fish skin graft resulted in faster re-epithelialization beginning at day 10 until day 28, especially on day 14, the difference between fish skin graft and fetal bovine dermis was significant. Fish skin graft resulted in an increase of blood flow and increase newly formed blood vessels. Fish skin graft promote a complete formation of the epidermis after 21 days. And fish skin graft triggered less inflammatory responses (lower foreign body, fewer inflammatory cells).

Although the findings of this study provide evidence of fish skin grafts being a preferred embodiment, a fetal bovine dermis (Primatrix) product could of course still be used as an effective skin substitute according to the present disclosure, and under some conditions or considerations, may also be a preferred embodiment of a skin substitute as contemplated in the current disclosure.

Further, even though this study was conducted on the non-colored version of the fish skin product without antimicrobial agent's MB and GV, the extent of the created wound was classified as deep partial thickness burn wounds which injured both the epidermis, dermis layers and are often complicated and lengthy to treat. The role of fish skin in this study is to act not only as a temporary coverage but also as a transient scaffold for long term healing. This study provides many important insights on scaffolding effects of the original fish skin.

The tissue-regenerating wound treatments of the present disclosure, and particularly tissue-regenerating wound treatments comprising fish skin as a skin substitute and having antimicrobial properties provided either by the coloring agent(s) or by further added active agents provide prevention of bacterial colonization of the subject device compared to other currently known skin substitutes.

The phrase “bacterial barrier” may be understood to mean that the broad-spectrum antimicrobials provide a barrier to bacterial penetration of the dressing as this may help reduce infection and ensure that the transient scaffold functions as intended.

Skin substitutes in a broad sense may be considered to be biodegradable tissues that get infiltrated by the body's own cells and then get integrated, absorbed or broken down. Most skin substitutes have low innate ability to fend off bacterial invasion and can get colonized if there are bacteria existing in the wound. Bacterial colonization of a skin substitute can cause it to break down more rapidly and make it less likely to harbor ingrowth of host's cells.

Of the 76 skin substitutes listed two other skin substitutes that provide some antibacterial effects, namely PriMatrix AG and PuraplyAM. However neither of these two have been found to have the same antibacterial spectrum and antifungal activity as the antimicrobial agents used in the tissue-regenerating wound treatments of the present disclosure, and particularly tissue-regenerating wound treatments comprising fish skin as a skin substitute and having antimicrobial properties provided either by the coloring agent(s) or by further added active agents. Of course, as noted above, PriMatrix AG and PuraplyAM may still be considered as embodiments of skin substitutes in the present disclosure, and indeed, under certain circumstances and conditions, may be preferred embodiments.

| Hydrophera | Puraply AM | Kroma AM | Primatrix AG |
|---|---|---|---|
| Bacillus subtilis | Aspergillus niger | Escherichia coli** | Escherichia coli |
| Escherichia coli | Candida albicans | Pseudomonas aeruginosa** | |
| MRSA | Staphylococcus aereus | Candida albicans* | Methicillin |
| | | | Resistant |
| | | | Staphylococcus |
| | | | (MRSA) |
| VRE | MRSA | Bacillus subtilis* | Vancomycin |
| | | | Resistant |
| | | | Enterococcus |
| | | | faecalis (VRE) |
| Serratia marcescens | Pseudomonas aeruginosa | MRSA* | Serratia |
| | | | marcescens |
| Staph aureus | Escherichia coli | VRE* | Staphylococcus |
| | | | aureus, |
| Staph epidermidis | | Serratia marcescens* | Staphylococcus |
| | | | epidermidis |
| Pseudomonas aeruginosa | | Staph aureus** | Acinetobacter |
| | | | baumanni |
| Pseudomonas florescnes | | Staph epidermidis* | Listeria |
| | | | monocytogenes |
| Enterococcus faecalis | | Pseudomonas aeruginosa* | Enterococcus |
| | | | faecium |
| Streptococcus pyogenes | | Pseudomonas florescnes* | Streptococcis |
| | | | pyrogenes (Group A) |
| Klebsiella pneumonaie | | Enterococcus faecalis* | |
| Proteus mirabilis | | Streptococcus pyogenes* | |
| Proteus vulgaris | | Klebsiella pneumonaie* | |
| Enterobacter aerogenes | | Proteus mirabilis* | |
| Yersinia enterocolitica | | Proteus vulgaris* | |
| Candida albicans | | Enterobacter aerogenes* | |
| Candida krusei | | Yersinia enterocolitica* | |
| Candida glabrata | | Candida krusei* | |
| | | Candida glabrata* | |
| | | Aspergillus niger* | |

Of course, as noted above, PriMatrix AG and PuraplyAM may still be considered as embodiments of skin substitutes in the present disclosure, and indeed, under certain circumstances and conditions, may be preferred embodiments. The antibacterial coverage of the subject device will be the comparable to Hydrofera Blue, which is a dressing that has an equivalent MB and GV concentration.

The tissue-regenerating wound treatments of the present disclosure, and particularly tissue-regenerating wound treatments comprising fish skin as a skin substitute and having antimicrobial properties provided either by the coloring agent(s) or by further added active agents may provide what may be term “device identification,” which promotes optimal utilization cycle. “Device identification” may be understood as the coloring agents making identification of the product easy when integrating into the wound bed.

Skin substitutes are most transparent or off white before application and become transparent, white or caramelized on integration into the wound bed. This appearance can be indistinguishable from wound slough, exudate or biofilm in some cases, especially for less experienced users. This makes it hard to determine if the skin substitute has fully integrated and needs replacement or if it is still partially active and can be kept in the wound longer. Inability to determine if there is still active skin substitute in the wound can lead to three possible outcomes: (1) Slough in wound mistaken for collagen dressing, which leads to the provider not removing slough from the wound and therefor slowing down wound healing and increasing risk of infection; (2) Active product in the wound mistaken for slough, which leads to provider removing the device prematurely; and (3) Removal of active transient scaffold tissue with fresh host cell ingrowth.

Active product in the wound mistaken for slough. Leads to early reapplication of device with an unnecessary intervention and associated cost for patient.

The novel device as disclosed herein is colored using biocompatible coloring agents that make it safe and easy to distinguish from slough or other tissue. This is done with the disclosed color agents that bind the color to the graft.

The device represents a breakthrough technology and a novel application of a technology that has the potential to lead to a clinical improvement in the treatment of chronic, non-healing wounds and in the prevention of possible amputations. The device offers a 3D structure to support human cells to infiltrate and proliferate, neovascularization while inhibiting bacterial colonization on the scaffold.

The tissue-regenerating wound treatments of the present disclosure, and particularly tissue-regenerating wound treatments comprising fish skin as a skin substitute and having antimicrobial properties provided either by the coloring agent(s) or by further added active agents will at least have one or more, of preferably all of the following characteristics: will provide a stable, resorbable scaffold that promotes cell ingrowth and neovascularization; will provide broad spectrum coverage to address microorganisms often present in wounds; not cause toxicity for host cells or inhibit cell ingrowth compared to silver-containing dressing; will not cause mutation of bacterial result in antimicrobial resistance compared to antimicrobial dressings.

Further, a color change might occur in the dressing by depletion of the coloring agents which could provide important a visual indicator to guide dressing change.

Applicant has multiple in vitro and in vivo transitory scaffolding data on previously cleared device Omega3 Wound. The inventors' evidence shows that the addition of the coloring agents (antimicrobial agents) to the scaffold does not interfere with the base functions and have synergistic additive effects.

In a in vitro study (Magnusson, Military Medicine, 2017) for cell ingrowth it was found that fibroblast infiltrate and remodel the fish skin graft after 12-days compared to hHACM material that had less infiltration of fibroblast. The tissue-regenerating wound treatments of the present disclosure, and particularly tissue-regenerating wound treatments comprising fish skin as a skin substitute and having antimicrobial properties provided either by the coloring agent(s) or by further added active agents will retain the same porous structure and pore size as the original fish skin which will attract cell infiltration into the scaffold. To address the toxicity of MB and GV to the cells, the inventors have performed preliminary cytotoxicity tests and found MB and GV but did not cause any cytotoxicity concern. Further, since a reference device, Hydrofera Blue Ready, contains higher concentration of MB and GV but did not cause any cytotoxicity concern, the tissue-regenerating wound treatments of the present disclosure, and particularly tissue-regenerating wound treatments comprising fish skin as a skin substitute and having antimicrobial properties provided either by the coloring agent(s) or by further added active agents should not cause any unfavorable effect to cell ingrowth.

Preliminary bench studies have demonstrated the efficacy of the tissue-regenerating wound treatments of the present disclosure, and particularly tissue-regenerating wound treatments comprising fish skin as a skin substitute and having antimicrobial properties provided either by the coloring agent(s) (MB/GV) on antibacterial capability. The test were done using the three most commonly found organisms in wound infections, E. coli, S. aureus and P. aeruginosa. The testing methods ranged from simple, basic assays such as the agar disk-diffusion to more challenge, industrial standardized tests such as AATCC100 or ASTM E2149. The results of the agar disk-diffusion showed that compared to Omega3 Wound and Primatrix AG, the tissue-regenerating wound treatments of the present disclosure comprising fish skin as a skin substitute and having antimicrobial properties provided either by the coloring agent(s) (MB/GV) exhibited a formation of distinct zone of inhibition for S. aureus. The inhibition zone diameter was 17.25±0.5 mm by the tissue-regenerating wound treatments of the present disclosure comprising fish skin as a skin substitute and having antimicrobial properties provided either by the coloring agent(s) (MB/GV), 11.67±0.58 by Primatrix AG while fish skin showed no effect thus having an inhibition zone the same diameter as the sample's diameter (6mm). The AATCC100-assessment results demonstrated a high efficacy of the tissue-regenerating wound treatments of the present disclosure comprising fish skin as a skin substitute and having antimicrobial properties provided either by the coloring agent(s) (MB/GV) with both S. aureus and P. aeruginosa. The reduction rate was estimated approximately 98% for P. aeruginosa. The tissue-regenerating wound treatments of the present disclosure comprising fish skin as a skin substitute and having antimicrobial properties provided either by the coloring agent(s) (MB/GV) showed a strong antibacterial efficacy against both S. aureus and P. aeruginosa. The ASTM E2149 test results demonstrated a drop on growth in E. coli suspension by Kroma Antimicrobial. There were 37 colonies formed on the agar plate with the tissue-regenerating wound treatments of the present disclosure comprising fish skin as a skin substitute and having antimicrobial properties provided either by the coloring agent(s) (MB/GV) while there were 445 and 491 colonies in the non-colored fish skin and E. coli suspension itself. The growth reduction rate was approximately 92-93% in favor of the tissue-regenerating wound treatments of the present disclosure comprising fish skin as a skin substitute and having antimicrobial properties provided either by the coloring agent(s) (MB/GV).

In view of the inventors' encouraging results from our preliminary testing, the tissue-regenerating wound treatments of the present disclosure comprising fish skin as a skin substitute and having antimicrobial properties provided either by the coloring agent(s) (MB/GV) will deliver a more effective antimicrobial treatment for various wound types.

The collagen scaffolds are widely used for chronic wound management as to enhance the wound healing process. Bioactive wound dressings has an advantage over other types of dressings as there biocompatibility and EMC-template like enhances cell ingrowth and tissue-regeneration.

The inventors have found and disclosed various embodiments, including a preferred embodiment using a fish skin transient scaffold combined with two antimicrobial coloring agents for the management of wounds an as an effective barrier to resist microbial colonization within the scaffold. The transient scaffold supports neovascularization and cell ingrowth while inhibiting microbial colonization of the dressing.

The tissue-regenerating wound treatments of a preferred embodiment of the present disclosure comprising fish skin as a skin substitute and having antimicrobial properties provided either by the coloring agent(s) (MB/GV) is an acellular resorbable fish dermal wound matrix. The wound treatment acts as a transient scaffold that supports neovascularization and cell ingrowth while inhibiting bacterial colonization on the scaffold. The device contains two antimicrobials agents that provide broad-spectrum antimicrobial protection with methylene blue and gentian violet (crystal violet) on the scaffold. The subject device is supplied as a sterile intact, or meshed sheet raging in sizes up to 20×30 cm. The broad-spectrum antimicrobials provide a barrier to bacterial penetration of the dressing as this may help reduce infection and ensure that the transient scaffold functions as intended.

Indications for Use

The tissue-regenerating wound treatments of preferred embodiments are intended as an antimicrobial transitory scaffold for the management of wounds including: diabetic foot ulcers, arterial ulcers, pressure ulcers, venous leg ulcers, and traumatic wounds.

Composition of the device

The tissue-regenerating wound treatments of preferred embodiments is fish skin medical device indicated for the management of wounds. The subject scaffold material, herebelow referred to at times a device, is obtained from the skin of wild North-Atlantic cod (Gadus morhua) by a standardized controlled manufacturing process and supplied in a peel-pouch terminally sterile packaging in the following sizes: 16 mm disc; 2×2 cm; 2×4 cm; 5×5 cm; 10×10 cm; 20×30 cm. The device may also be provided in particalized form, as described and shown above.

The device may contain two antimicrobials agents, such as methylene blue and gentian violet (crystal violet), that provide broad-spectrum antimicrobial protection with on the scaffold. The concentration of MB and GV is controlled at equal or less than 0.00025 g/g (0.01%), but may include up to 0.1% or less.

The subject device preferably becomes completely integrated into the surrounding tissue over time, corresponding new host tissue deposition. Preferred physical properties of the subject device allow cellular ingrowth. The subject device is preferably biocompatible, non-crosslinked and bioresorbable, strong, and pliable. Its tensile strength supports fixation by sutures or staples.

The subject device mechanism of action can be broken down into three main domains: 1. Collagen Dressing: Substantially equivalent to the decellularized fish skin Wound product (K132343), with the following differentiating properties: 1.1. It is impregnated with antimicrobial coloring agents that: 2a: “Bacterial Barrier”: The broad-spectrum antimicrobials provide a barrier to bacterial penetration of the dressing as this may help reduce infection and ensure that the transient scaffold functions as intended. 2b: “Device identification” The coloring agents make identification of the product easy when integrating into the wound bed. 2. “Transient Scaffolding” A transient scaffolding, supporting cell ingrowth, neovascularization, and the regeneration of extracellular matrix. 2.1 Collagen dressing. The subject device functions substantially equivalent to decellularized fish skin Wound product as a collagen dressing with the same underlying mechanism of action.

The main purpose of a collagen scaffold is to serve as template to mimic the extracellular matrix (EMC) of healthy tissue. By mimicking to support cells to aid the reconstruction of many different tissue types to help in the wound healing process. Each component of the EMC is essential for each of the phases of wound healing. Components of the ECM play key roles in aiding cell proliferations and differentiation, guiding cell migration, and modulation cellular responses. Exogenous EMC's will undergo the natural remodeling of healthy tissue in the wound site as it is degraded and replace by native collagen. Decellularized fish skin Wound product re-establish a functional EMC in chronic wounds. The collagen dressing also offers: (1) Moist wound environment, (2) Fluid management, and (3) Transpiration control of fluids.

Decellularized fish skin wound product may be used as a collagen dressing as substantially equivalent to many of the porcine collagen matrixes that had been cleared through 510k premarket notification process. The efficacy of the device as a collagen dressing for the management of wounds proved by a non-inferiority study comparing it to Oasis Wound Matrix, a mammalian-derived collagen dressing. The study concluded that the fish collagen dressing was not inferior to porcine sourced collagen dressing, no adverse reactions were identified, and that improvement of wound healing was found during a period of 28-weeks.

The only technical difference between decellularized fish skin wound product and the subject device is the addition of coloring agents. No evidence or literature evidence where either of the coloring agents disrupts or cross-links with collagen scaffolds. Therefore, based on the inventors evidence, there enough evidence to suggest that the subject device, whether a fish skin based skin substitute, or other skin substitutes, will also serve as a scaffold to mimic the extracellular matrix to aid in cell ingrowth and neovascularization.

The colorants of the preferred embodiments have significant antimicrobial effects. Using coloring agents of approximately 0.01% of a mixture of Methylene Blue (MB) and Gentian Violet (GV), the two antimicrobial agents provide broad-spectrum antimicrobial protection against both gram-negative and gram-positive bacteria. When in contact with bacteria, MB and GV in the subject device will eliminate bacteria by making the bacterial growth within the device unsustainable.

Methylene blue is part of the phenothiazine family and is one of the first FDA-approved therapeutic agents against malaria when resistance to antimalaria drugs occurred. MB has shown its bacterial inactivation with a wide range of organisms including E. coli, S. aureus, P. aeruginosa, and C. albicans in vitro.

The pigments of MB and GV works as an indicator to distinguish the device and slough in the wound. Since the skin substitute is a resorbable dressing, the color will notify physicians when the dressing is fully integrated, and a second application is needed. Also, the color will help reduce dressing removal by mistake when the physicians perform debridement on the wounds. Uncolored collagen dressings can sometimes be hard to distinguish from slough when partially integrated.

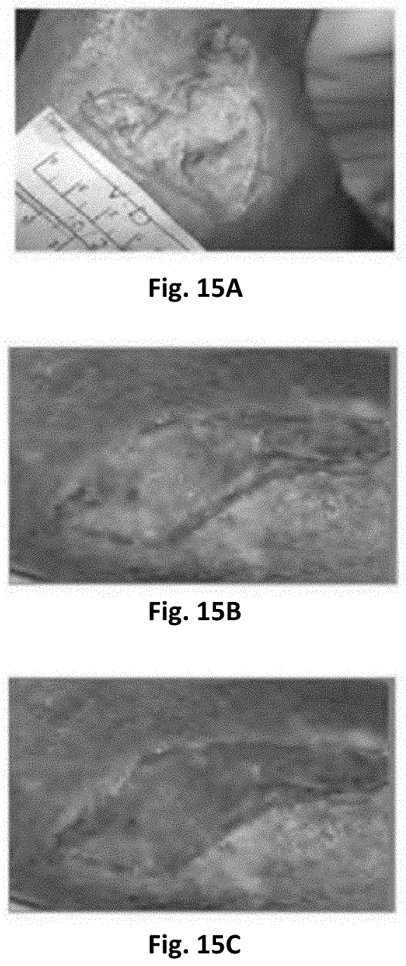

For example, FIG. 15A shows a graft in a wound that has turned to a slough in the wound that is full of bacterial. By comparison, FIG. 15B shows a fish skin graft within a wound that is approximately 50% integrated and should remain in the wound. However, as can be seen in comparing the slough within the wound of FIG. 15A as compared to the graft in the wound of FIG. 15B, it is difficult to correctly and easily distinguish the graft that is experience ingrowth from the graft that has become a slough. By comparison, FIG. 15C shows a skin substitute, in this case, a fish skin graft, that has been colored with the coloring agents of MB/GV. It is clear from that the graft of FIG. 15C is being integrated and ingrowth is occurring, and that the graft of FIG. 15C should remain for another week and should not be removed.

In vitro study adding mouse embryo fibroblast on top of the fish skin has shown the skin scaffold is highly porous and the cells were able to migrate and proliferate into the scaffold. In an animal study, the fish skin was applied on burn wounds generated on a porcine model. The fish skin graft resulted in faster wound heal and showed a superior blood flow under the fish skin as well as an increase in newly formed blood vessels. In the same porcine study, the fish skin graft promoted a complete formation of the epidermis after 21 days with faster re-epithelialization and less inflammatory response.

When applied to the patient's wound, embodiments of the subject device rapidly integrates into the wound, provides a transient scaffold for cell migration and proliferation while the MB and GV molecules inhibit and eliminate microbial colonization on the matrix. The enriched dermal collagen fibers support cellular ingrowth, neovascularization, and regeneration of the extracellular matrix which are critical to faster wound healing.

The colored skin substitute, for example, fish skin graft is eventually broken down by the body. Cell ingrowth of primary fibroblasts with some inflammatory component in the end completely remodel and break down the original skin substitute, such as the fish skin graft, and the colors.

The enzymatic process is primarily hydrolysis of the collagen into smaller and easier process particles and reduction of the coloring agents.

The colored skin substitute, for example, fish skin graft may have the following characteristics: up to 7×20 cm, or even 20×40 cm, may be Solid or Meshed, and may be in sheets or particalized.

Further Examples of Production of the Wound Treatment

In yet a further example of a method or procedure for production of an embodiment of the tissue-regenerating wound treatment the follow procedure was followed.

Ten skins were flat frozen in a pack prior to our arrival.

A coloring solvent was mixed and prepared including 0.01% and a 0.005% dilution of MB&GV.

In order to have clean water, a sink was used which was monitored for bacterial amount and boiled it before using.

First a 1% stock solution was created.

GV: 650 mg pharmaceutical grade (USP), SA-1290002, LOT G1K417, SP1098511 (Distica)

MB: Methylene Blue hydrated for microscopy, ≥97%.0%, Sigma-Aldrich 66720-100g, LOT #BCBZ4929

0.4g MB+0.4g GV were added to 40 ml clean (still warm) water in a boiled flask. Note that 250 mg of GV is left.

Two 2L bottles were filled with 2L of clean/boiled water (measured by weight). From one bottle, 10 ml was removed with a clean pipette, from the other, 20 ml water was removed. These volumes were replaced with the stock solution, to create 0.005% and 0.01% MB&GV solutions, respectively. The water in the bottles was quite hot to the touch once the final solutions had been prepared, which might affect the results of the staining.

Note that the two compounds stained all surfaces extensively, meaning vigorous cleanup of all surfaces was needed, with water and ethanol.

The fish skins were then removed from the freezer and placed on ice to be transported along with the coloring solution. All was brought into the high risk area chamber next to the freeze dryer. Fish skins were thawed under flowing tab water from a high risk chamber faucet. Once they were soft and pliable, they were cut in two shorter pieces, as the skins were quite large and long. 5 fish skins (10 halves) were placed into two aluminum trays for staining.

Around 700 ml Kroma solution was placed into each trays, labeled 0.01 and 0.005% respectively. The two trays were then placed into plastic bags, which were folded to reduce risk of spillage, and placed on a shaker sett at 30 rpm for 2 hrs.

After around 20-30 minutes, the skins were moved around with a pair of sterile pliers in order to encourage an even staining. Around 20 min later, it became apparent that the staining solutions were losing density and becoming clearer, as the fish skin absorbed the stain. Around 300 ml of staining solution was added to each tray to correct for this, meaning the final staining volume was around 1000 ml.

A portion of both the original coloring solutions and the leftover of the used staining solutions will be saved in 50 ml tubes in order for possible concentration quantification later. This, in combination with measurements on the fish skin sizes and weight post freeze drying, might enable us to roughly quantify the absorption of color into the fish skins.

The freeze dryer was started just after 18:00, as it takes around 45 min to become ready for a run.

The freeze dryer had some difficulties starting due to computer error. Therefore the staining took around 3 hrs (note that the shaker returns to default shaking speed after 3 hrs, which is faster). The skins were thoroughly washed with running tab water in the high risk room for 10-15 min.

The remaining coloring solution had definitely cleared somewhat again, and there was a slight color difference in the two batches of fish skin: the 0.01% was a true denim dark blue, while 0.005% was more like a medium denim blue. Samples of the remaining staining liquid were collected in 50 ml tubes in case quantification is possible. Both batches took around 1 and a ½ plate, so in total we had three full plates. The stronger dyed skin was on the left hand side of the shared plate.

Freeze drying was started at around 8:30 in the evening, and left to run over night.

The fish skin was freeze dried in the morning, and packaged for non-sterile uses. The (roomtemp) leftover 0.01 and 0.005% solutions were used to repeat another batch the next day.

Further Examples of Production of Prototype Wound Treatment

To produce two prototypes of Colored Cod skin of two different concentration of the Methylyne blue and Gentian Violet. The concentration of the color solutions are 0.01% w/v aqua solution w/v; Methylyne Blue (50%) and Gentian Violet (50%) and 0.005% w/v aqua solution w/v; Methylyne Blue (50%) and Gentian Violet (50%).

Material: 10 Cod Skins descaled and decelled Batch DC 21039A; 1 Liters 0.01% w/v aqua solution w/v; Methylyne Blue (50%) and Gentian Violet (50%); 1 Liters 0.005% w/v aqua solution; Methylyne Blue (50%) and Gentian Violet (50%); 10 aluminum trays; Scissors; Small Tyvek pouches; Big Tyvek pouches; Big Plastic bags; Shaker; Sealer.

Prototype process: All the cod skins were fresh from the manufacturing production the same day. Frozen at −80 C for 5 hours. The cod skins were too big to fit into the aluminum trays and were therefore cut into two pieces giving total of 20 pieces of cod fish skins.

Prototype 0.01%

1 liter of the 0.01% solution poured into a aluminum tray marked MB-GV 0.01%, 10 pieces of cod laid evenly into the tray ensuring that the solution covered the skins. The tray put into a plastic bag to minimize risk of spilling the color, then the tray was placed onto the shaker at pro:40 for 3 hours.

Prototype 0.005%

1 liter of the 0.005% solution poured into a aluminum tray marked MB-GV 0.005%, 10 pieces of cod laid evenly into the tray ensuring that the solution covered the skins. The tray put into a plastic bag to minimize risk of spilling the color, then the tray was placed onto the shaker at pro:40 for 3 ½ hours.

Start of coloring on shaker: 15:40±10 min.

End of coloring on shaker: 19:05±5 min.

Rinsing start at: 19:05±5 min.

Rinsing stop at: 19: 20±5 min.

Freeze drying: All the fish skins were stretched out on the steal plates and sandwiched with another plate placed on top. Freeze dryer program: SvavaColor- total time of 10 hours.

Packaging: Visual inspection and bending tests supported clean and dry colored cod fish skins, ready for packaging. The samples were packed into Tyvek pouches, small and big samples pouches, marked and sealed.

No scraping of the skins was done on these prototypes.

Crosslinking to Improve Color Fastness and Mechanical Properties of Wound Treatment

In further embodiments, it has been found by the inventors that crosslinking of the skin substitute, for example, of a scaffold material, can further enhance the propreties of the skin substitute, including increasing the fastness of the coloring agent that colors the skin substitute, increasing the mechanical material properties of the skin substitute, increase resistance to enzymatic and chemical degradation in the skin substitute, and enhance the lifetime of the coloring agent added to the skin substitute in biological conditions, such as in a treated wound. In preferred embodiments, the main objective for crosslinking the skin substitue, for example, a scaffold material, is to have a colored product that maintains its color for at least one day after being applied to a wound, and preferably three days after being applied to a wound, and even more preferably up to 8-10 days, even preferably still, up to 14 days after being applied to the a wound.

As described herein, crosslinking of the skin substitute and/or skin substitute with added coloring agent can be performed by various means, for example, by irradiation or by chemical means.

Chemical Crosslinking or Modifiers

In one embodiment, the skin substitute is crosslinked by treatment of the skin substitute with a crosslinking agent. In another embodiment, proteins of the skin substitute are otherwise modified by treatment of the skin substitute with a protein-modifying agent.

In embodiments, the chemical crosslinking agent targets one or more of the following groups: primary amines (—NH2); carboxyls (—COOH); sulfhydryls (—SH) or carbonyls (—CHO), or some other group. Thus crosslinking agent may be, for example, amine-reactive, carboxyl-to-amine reactive, sulfhydryl-reactive, and/or aldehyde-reactive.

In a first embodiment, the cross linking agent is a simple sugar or monosaccharide. Alternative sugars may be used, including glucose, fructorse, or galactose. For example, the crosslinking agent may be or include ribose. Alternative sugars may be used, including glucose, fructorse, or galactose. Compound sugars, disaccharides, may also be contemplated.

In another embodiment, the crosslinking agent is natural or synthetic crosslinking agent. For example, in an embodiment the crosslinking agent includes or is genipin.

### Example 1

### Ribose Crosslinking

A first example is provided herein wherein ribose is used as a crosslinking agent.

According to this example, a standard ribose (stock) solution is prepared. For this a 0.2 M (molar) solution of ribose was made in PBS containing 0.05% (w/v) sodium azide to prevent bacterial growth. Other concentrations of the ribose may be used, and other bacterial-growth preventing agents may be used. In this example, by weight the solution includes 30.03 g ribose, 9.55 g premixed PBS standard, and 50 mg sodium azide. The dry ingredients are weighed using a precision scale added to a 1L volumetric flask and diluted to point, 1.00 L, with deionized water. The ingredients are mixed until completely dissolved and then the solution is ready to use.

In this example, a Kerecis' fish-skin-derived cellular scaffold product is used as the skin substitute. In general, any size and/or number of scaffolds could be treated, even including particalized scaffold material. The container in which the scaffold material is added to the solution may be large and the volume of ribose solution completely covers the samples. In this example, the scaffold material were cut to pieces of 4×8 cm². Five (5) pieces or samples were cut form a larger sample, so the longer side (8 cm) was in parallel to the length of the cod fish skin. The samples were then submerged in roughly 250 mL of the ribose solution for between 3 and 6 days at room temperature. A first sample was removed on the 3-day mark, the next two at 5 days, and the last two at 6 days. An additional sample of the same size was also prepared by submersing it in roughly 80 ml for 40 hours.

After each sample was removed from the ribose solution it is washed with running water and then put in a water bath for two days, to wash out any unreacted ribose along with PBS and sodium azide. The water is additionally changed periodically (once or twice each day) to aid the washing process. The samples are then partially dried and frozen for further processing.

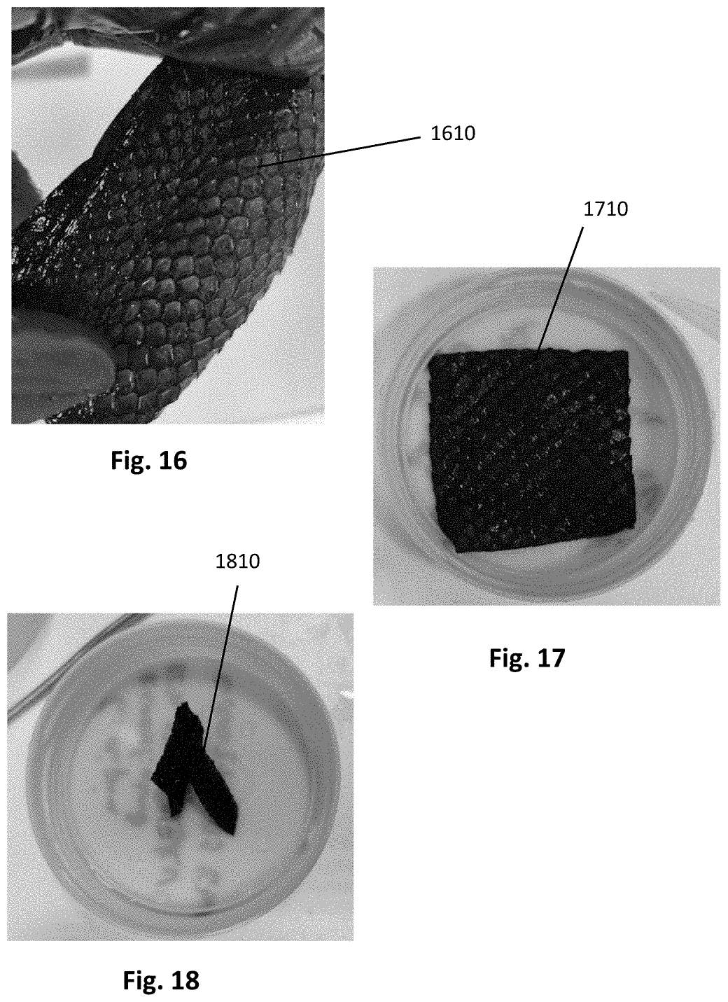

Methods of dyeing for ribose-crosslinked scaffolds: Two general methods have been used to dye crosslinked scaffolds. The first could be described as meta-dyeing, where MB and GV are added to the crosslinking solution. For this method one piece of 4×8 cm² scaffold was submerged for 24 hours in a solution consisting of 98 mL standard/stock ribose solution (same as described above) and 1 mL of each dye (MB/GV) stock solution, the stock solutions are 0.1 wt %, so the concentration of MB/GV in solution is 0.002%. After the combined crosslinking/dye process the sample was washed in the same way as described above for crosslinking, first under tap water and then left in water for two days, to produced sample 1610 as shown in FIG. 16. Sample 1610 of FIG. 16 is a meta-dyed, ribose crosslinked and dyed scaffold left for 24 hours in “meta” solution.

According to the second method, post dyeing uses the same conditions as have been discuses in previously for the “standard dye process”, i.e., after the scaffold has gone through the crosslinking and washing process the sample is dyed for 3 hours in a 0.002 wt % solution of MB/GV in PBS, for this a 4×4 cm² piece of scaffold was dyed in 100 mL of solution. This can be done with a pre-crosslinked scaffold regardless of the crosslinking time e.g., 24 hours, 40 hours, 5 days, or 6 days. FIG. 17 shows a post-dyed, ribose crosslinked scaffold 1710 left for 40 hours after a 3-hour standard dye prossess in 0.002% MB/GV PBS solution.

Other embodiments and examplary methods may include variations from the above ribose cross-linking examples. The process of meta-dyeing can be changed in at least two ways. A first change may include increasing or decreasing the time in solution. The second may include changing the concentration of either the dye or ribose in solution. As the absorption of dye happens relatively slowly over time and is directly linked to the dye concentration of the solution, it may, for example, be productive to decrease the concentration of MB and GV in solution if the meta-dyeing was, for example, 48 hours, if the concentration of colour in the scaffold should be the same as for the 24 hour process described above, in practice any combination of time and concentration (within reason) is possible and would yield a unique result.

Regarding post-dyeing as stated above, the crosslinking time of the scaffold can be changed. If a change in concentration of the dye in the scaffold in effected, the concentration of dye and/or the time in dye-solution can also be changed.

### Example 2

### Genipin Crosslinking

In a second example, as noted above, genepin is used as a crosslinking agent, for which an examplary procedure is here described.

The genepin crosslinking solution is prepared. According to this example, a 0.3% (w/v) solution of genipin in PBS is made, 200 mL of the solution was made by dissolving 0.60 g of genipin in 200 mL of pre-made PBS solution (9.55 g premade PBS powder/1 L). The solution is stirred until no solid particulates remain.

In this example, a Kerecis' fish-skin-derived cellular scaffold product is again used as the skin substitute. In general, any size and/or number of scaffolds could be treated, even including particalized scaffold material. For this example, a culture plate with 15 mL wells was used. A 2×2 cm² piece of scaffold/collagen was placed in each well and the genipin solution is added. Each well was filled completely with 15 mL of total solution. The plate was then closed with the lid and sealed before being submerged in a 37° C. water bath for 24 hours. Note that any other heating source could work if the temperature is consistent at 37° C. and evaporation is limited by sealing the vessel or recondensing the solution. Once the 24 hours in solution passed the scaffolds were washed with water and frozen. In this example, crosslinking with genipin turned the scaffolds blueish black in addition to causing the scaffold material to roll up. There was also a distinct difference in the stiffness of the sampless.

Similar to ribose crosslinking and dyeing, the two methods that have been explored with this example are dyeing during the crosslinking and after, i.e., meta and post dyeing. In this example, six wells were used, four of which only contained 15 mL of the 0.3% genipin solution and two containing MB and GV as well (meta-dyeing). The meta-dyeing solutions were prepared by adding 150 μL of each dye stock solution (0.1 wt %) to the well and then adding 14.7 mL of the genipin solution. Except for the addition of MB/GV, all six wells were the same and got the same treatment during the crosslinking process.

The post-dyeing procedure for genipin crosslinked scaffolds is the same as for ribose crosslinked scaffolds. The sample is submerged in a 0.002% MB/GV, PBS solution for 3 hours, for each 2×2 cm² that is dyed 25 mL of solution is used. Here 2 pieces of 2×2 cm² scaffold were dyed so 50 mL of solution was used to produced the sample 1810 in FIG. 18. FIG. 18 shows sample 1810, which is a post-dyed, genipin scaffold, dyed in 0.002 wt % MB/GV PBS solution for 3 hours. After the dyeing the samples were washed, partially dried and frozen.

Other embodiments and examplary methods may include variations from the above genipin cross-linking examples. The changes that could be made to the dyeing process of genipin cross-linked scaffold samples are in essence the same as for the ribose method described earlier. That is that changes can be made to the time in and concentration of the dye regardless of the process (meta- or post-).

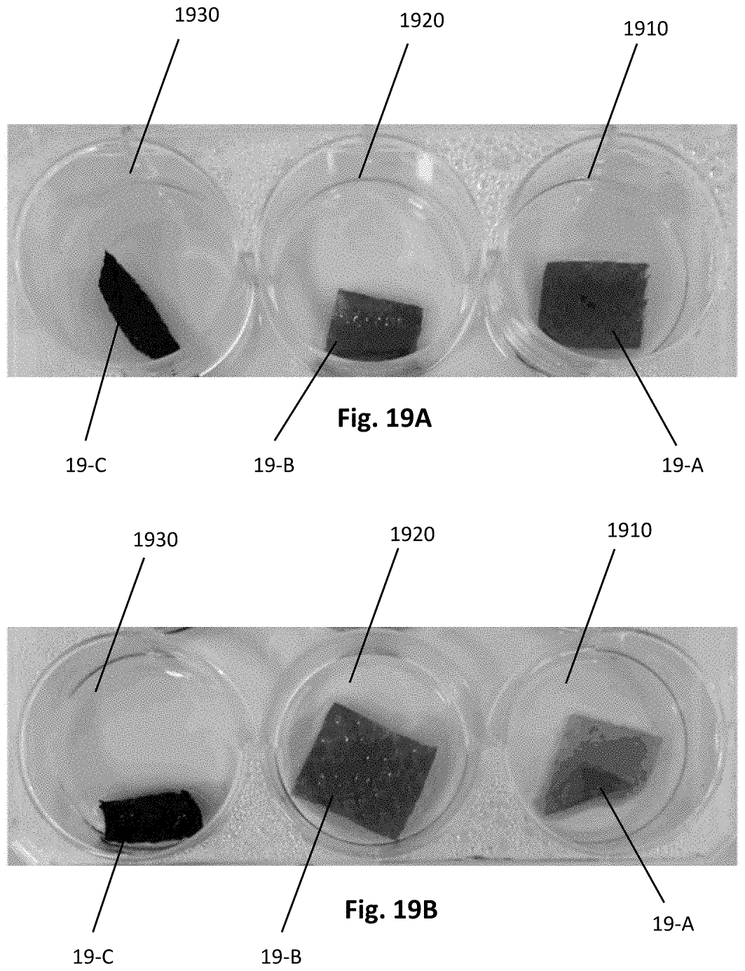

FIGS. 19A and 19B show a comparison of the improved maintenance of the coloring due to chemical crosslinking. FIG. 19A shows a comparision of pieces 19-C, 19-B, and 19-A in dishes 1930, 1920, and 1910, respectively. Each of the samples from which pieces 19-C, 19-B, and 19-A were taken was a Kerecis™ fish-skin-derived cellular scaffold product that in a 0.002% MB/GV, PBS solution for 3 hours. The sample from which piece 19-C was taken was also crosslinked with a 0.3% genipin solution according to Example 2 above. The sample from which piece 19-B was taken was also crosslinked with a ribose solution according to Example 1 above. And the sample from which piece 19-A was taken was not crosslinked, but only colored in a 0.002% MB/GV, PBS solution for 3 hours.

For a comparison, FIG. 19A shows pieces 19-C, 19-B, and 19-A in dishes 1930, 1920, and 1910, respectively, after dyeing, and in samples for 19-C and 19-B, after crosslinking. Subsequently, a bicarbonate solution with a pH of 8 was added in equal amounts and equal concentrations to each of dish 1930, 1920, and 1910. Pieces 19-C, 19-B, and 19-A were kept in dishes 1930, 1920, and 1910, respectively for 48 hours at a tempearture of 37° C., resulting in the same pieces 19-C, 19-B, and 19-A after 48 hours as shown in FIG. 19B.

As can be seen, the color fastness of pieces 19-C and 19-B, which were respectively crosslinked with genipin (19-C) and ribose (19-B) was markedly improved over piece 19-A, which had been similarly colored but which had not been crosslinked. Crosslinked pieces 19-C and 19-B clearly show their coloring to be more fast and better maintained.

Additionally, it is worth noting that in both cases, for ribose and genipin, the concentration and time for the crosslinking process can be changed as well. This would affect the final colour for both procedures when meta-dyeing but would have a much greater effect in the case of genipin, as the colour that results directly form the crosslinking is extremely dark and concentrated when using the method as described above. If the time in solution, concentration or temperature would be lowered that would result in less crosslinking and a lighter colour as has been shown by several studies.

Crosslinking by Irradiation

In another embodiment, the skin substitute is crosslinked by irradiation of the skin susbtitute material with electromagnetic radiation. In a first example, the skin substitute, for example, a scaffold material, is irradiated with ultraviolet (UV) radiation.

Example of UV Crosslinking

According to a UV-based example, a 0.1% stock solution of Methylene Blue (MB) was prepared by adding 200 mL of sterile water to 200 mg of MB and stirring until the color dissolved. A PBS solution was also prepared by mixing one liter of liquid 10× PBS with 8.8 liters of tap water and stirred.

A Kerecis' fish-skin-derived cellular scaffold product was again used as the skin substitute. In general, any size and/or number of scaffolds could be treated, even including particalized scaffold material, which may be as small as having a diameter of 1 mm. For this example, the pieces used included 14 pieces of 4×8 cm cut fish skin and two uncut fish skins. The fish skins were all pre-scraped to remove flesh tissue, scales, and fascia.

PBS and part of the stock solution were put in a large container and stirred until uniform. The amount of each solution was: PBS stock, 8.8 L; stock color, 200 mL.

After the color solution had been prepared, the skins were added to the color solution and stirred and it was ensured that no skins were stuck together.

The skins sat in the color solution for 3.5 hours and were stirred every hour. A UV cabinet was set up and the skins were arranged into trays.

The UV radiation source was a15W UV light (254 nm), which was screwed inside the cabinet so the trays could sit under the light during the radiation step. The interior of the cabinet was covered in aluminum foil to try and redirect the light of the wall on the skins. The trays were arranged to be approximately 12 cm from the light. Although UV light that was nearly monochromatic UV radiation was selected in this example, other UV sources of varying wattage and wavelength may be used in other embodiments, with either monochromatic UV radiation or polychromatic UV radiation, with the UV radiation having a wavelength within the range of about 10 nm to about 400 nm.

Samples were thus obtained including: Sample A, which was was removed from the MB-based coloring solution and placed in a PBS solution (without color) and was exposed to the UV radiation: Sample B, which was placed in the MB color solution and was exposed to the UV radiation while in the MB color solution; and Sample C, which was placed in the color solution but was not exposed to the UV radiation. In other words, skins of Sample A could be considered analogous to the post-dyeing crosslinking, in that the skins of Sample A were dyed in the MB-based color solution, and then the skins of Sample A were crosslinked by UV radiation while only in a PBS solution. By comparision, the skins of Sample B may be considered analogous to meta-dyeing in that the skins of Sample B remained in the MB-based coloring solution while being crosslinked by the UV radiation. And skins of Sample C may be considered a control in that the skins of of Sample C were dyed in the MB-based color solution, but were not exposed to the UV radiation, either during or after they were dyed. But as a control, the skins of Sample C were maintained in the UV cabinet, however, in a darked portion of the cabinet where they were not exposed to the UV radiation. In this way, the skins of Sample C were treated under similar temperature, flipping, and time conditions for the comparison with the crosslinked skins of Sample A and Sample B.

The skins sat in their respective liquid solutions for 6 hours. The skins floated at the top so the skins were flipped upside down to ensure that both sides were exposed to UV light equally. This was also done for the skins in the trays not exposed to UV light (in the dark) to have the process as much alike as the skins under UV light. When turning the skins, the UV light was turned off for approximately 5-10 min.

The temperature of the liquid solution was measured when turning the skins. The maximum temperature after 5 hours, was less than 25° C., so it was concluded that the UV light did not heat the solution and the skins to a degree where a cooling system was needed.

After the radiation step, the skins were collected from the trays and moved to three separate bags, one for each of Sample A, Sample B, and Sample C. The skins were rinsed for a few minutes in cold water and arranged on a steel plate before being inserted into the freeze dryer. The skins were in the freeze dryer overnight.

The skins were collected in three separate bags and each piece was sealed in a Tyvek bag.

Some pieces of the various samples were sterilized using Ethylene Oxide.

Sample A: The skins of Sample A were colored with the MB color solution, from whcih they were subsequently removed, rinsed, and placed in the PBS solution while under the UV light. The PBS solution originally had no color, but at the end of the UV radiation, it was clear that color from the skins from the coloring before had leaked from the skins and colored the PBS solution. The final color of the skins was more light blue and even a little green color on the skin compared to the other prototypes that were more dark blue.

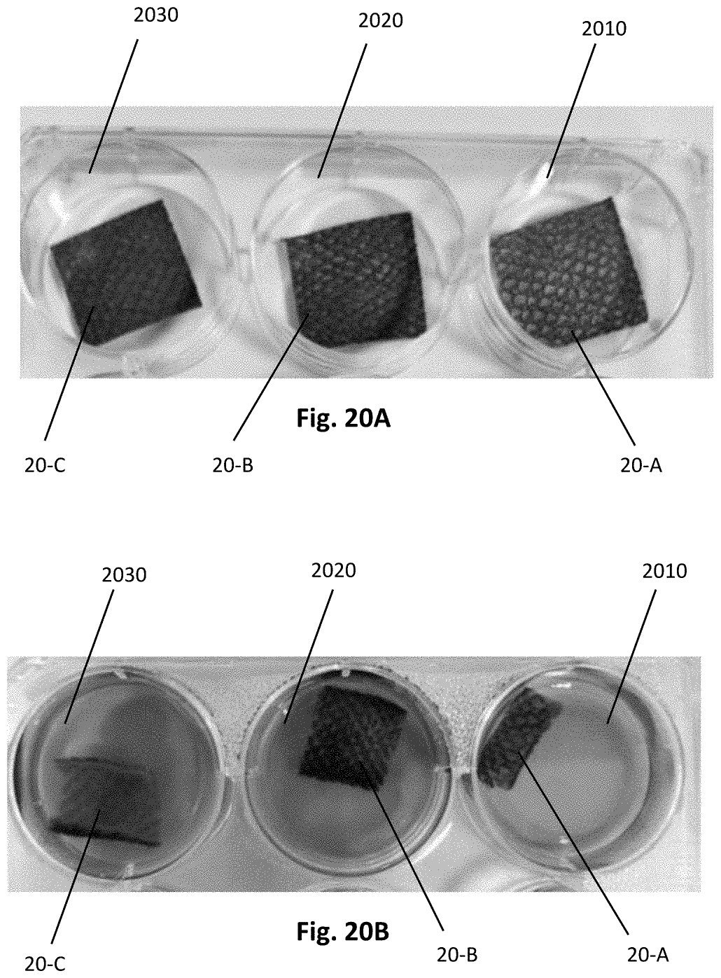

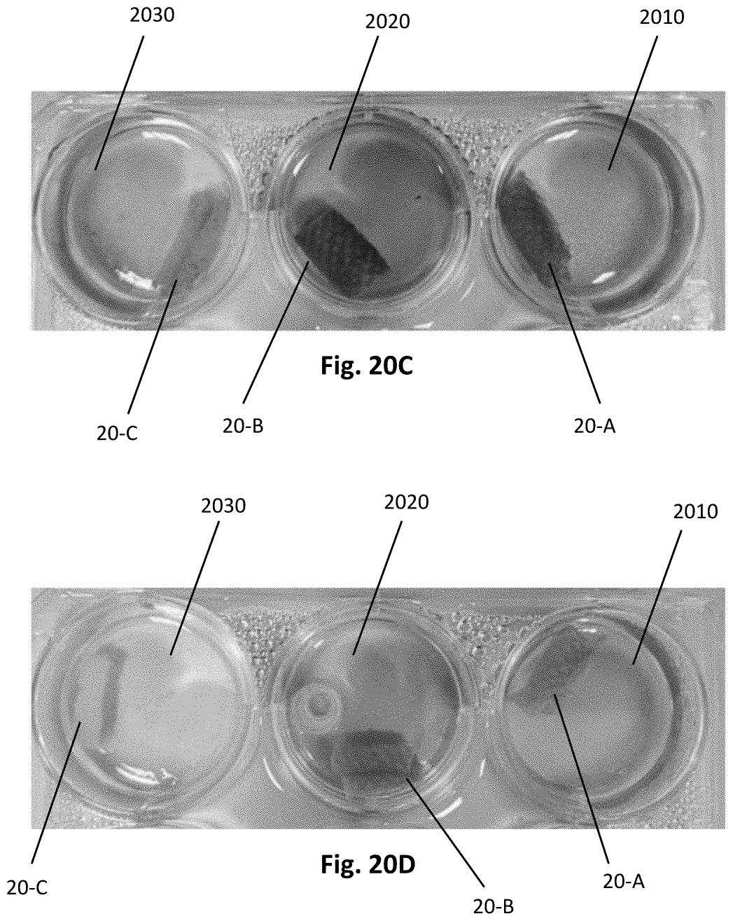

Samples B and Samples C: Upon finishing the crosslinking of the skins of Sample B, there was no visible difference between the colored skins of Sample B that sat under the UV light (Sample B) while in the MB-based color solution compared to the skins of Sample C, that were not exposed to the UV light, as can be seen in FIG. 20A, comparing a piece 20-B of Sample B with a piece 20-C of Sample C. A piece 20-A of Sample A is also provided in FIG. 20A for comparison. The skins of Sample B and Sample C had very similar color and there was no visible difference or feel to the texture of the fish skins.

Samples of the liquid solutions were not collected for color quantification. The prototypes were only made for testing the UV radiation and how it affects the fish skin.

Other methods might be used later for measuring the color quantity in the skins by breaking a part of the fish skin down in enzymes and measure the color quantity of that solution.

As a means to compare the color fastness of the crosslinked samples, the color leakage of a piece of each sample was performed by letting the samples sit in a base and acid (acid/base) solution at 37° C. Additionally, the samples were compared to other prototypes, previously made different mordants, colored while gradually changing the pH of the solution. The result showed that the samples crossliked with UV maintained the color for longer than many other prototypes.

FIGS. 20A to 20D show a comparison of the improved maintenance of the coloring due to crosslinking by UV irradiation. FIG. 20A shows a comparision of pieces taken from Sample C, B, and A, including a piece of Sample C, labeled as 20-C and placed in dish 2030; a piece of Sample B, labeled as 20-B and placed in dish 2020; and a piece of Sample A, labeled as 20-A and placed in dish 2010. FIG. 20B shows pieces 20-C, 20-B, and 20-A in dishes 2030, 2020, and 2010, respectively, after being in the acid/base solution of the same concentration for 24 hours. FIG. 20C shows pieces 20-C, 20-B, and 20-A in dishes 2030, 2020, and 2010, respectively, after being in the acid/base solution of the same concentration for 48 hours. And FIG. 20D shows pieces 20-C, 20-B, and 20-A in dishes 2030, 2020, and 2010, respectively, after being in the acid/base solution of the same concentration for 72 hours. As can be seen, the color fastness of pieces 20-B and 20-A, which had been exposed to UV radiation was markedly improved over piece 20-C, which had been similarly colored but which had not been explosed to the UV radiation. This is particularly the case as can be seen in FIGS. 20C and 20D, at 48 hours and 72 hours, respectively in the acid/base solution. After both 48 and 72 hours, piece 20-B and 20-C still maintained some color, while after 72 hours uncrosslinked piece 20-C had become nearly white or returned to its original color. A comparison of pieces 20-B and 20-A at both 48 hours and 72 hours shows that the meta-dyeing of Sample B, wherein the fish skins were dyed with the UV radiation while being within the MB-based color solution appears to provide a slightly more color-fast fish skins when exposed to the acid/base solution. That is, the color of piece 20-B appeared to be slightly darker than piece 20-A after both 48 and 72 hours of being in the acid/based solution.

Other embodiments and examplary methods may include, but are not limited to, variations of the coloring agent, the intensity of the UV radiation, variations in the wavelength or range of wavelength of the UV radiation, variations in the dyeing times, variations in the dyeing concentrations, and variations in the time exposed to the UV radiation.

Based on these examples and described embodiments, it has been found and shown by the inventors that the properties of a colored skin substitute, for example, a colord scaffold material, can be improved including increasing the fastness of the coloring agent that colors the skin substitute, increasing the mechanical material properties of the skin substitute, increase resistance to enzymatic and chemical degradation in the skin substitute, and enhancing the lifetime of the coloring agent added to the skin substitute in biological conditions, such as in a treated wound.

Combinability of Embodiments and Features

This disclosure provides various examples, embodiments, and features which, unless expressly stated or which would be mutually exclusive, should be understood to be combinable with other examples, embodiments, or features described herein.

In addition to the above, further embodiments and examples include the following:

1. A tissue-regenerating wound treatment comprising: a skin substitute; and a coloring agent added to the skin substitute, the coloring agent being a biocompatible coloring agent that degrades upon protease attack within a treated wound.

2. The tissue-regenerating wound treatment according to any or a combination of 1 above or 3-14 below, wherein the skin substitute is a biological skin substitute, or synthetic substitute, or a hybrid of biological and synthetic skin substitutes.

3. The tissue-regenerating wound treatment according to any or a combination of 1-2 above or 4-14 below, wherein the skin substitute is an autologous skin graft, a syngeneic skin graft, an allogeneic skin graft, a xenogeneic skin graft, or a synthetic skin graft.

4. The tissue-regenerating wound treatment according to any or a combination of 1-3 above or 5-14 below, wherein the skin substitute includes a scaffold material.

5. The tissue-regenerating wound treatment according to any or a combination of 1-4 above or 6-14 below, wherein the skin substitute includes a scaffold material that includes an extracellular matrix product.

6. The tissue-regenerating wound treatment according to any or a combination of 1-5 above or 7-14 below, wherein the extracellular matrix product is in the form of particles, or a sheet, or a mesh.

7. The tissue-regenerating wound treatment according to any or a combination of 1-6 above or 8-14 below, wherein the skin substitute is a scaffold material comprising intact decellularized fish skin, wherein the intact decellularized fish skin comprises extracellular matrix material.

8. The tissue-regenerating wound treatment according to any or a combination of 1-7 above or 9-14 below, wherein the wound treatment is crosslinked, before, after, or while the coloring agent is added to the skin substitute.

9. The tissue-regenerating wound treatment according to any or a combination of 1-8 above or 10-14 below, wherein the coloring agent includes a thiazine dye, or a triarylmethane dye, or a combination of a thiazine dye and a triarylmethane dye.

10. The tissue-regenerating wound treatment according to any or a combination of 1-9 above or 11-14 below, wherein the coloring agent includes methylene blue (MB), or gentian violet (GV), or a combination of methylene blue (MB) and gentian violet (GV).

11. The tissue-regenerating wound treatment according to any or a combination of 1-10 above or 12-14 below, wherein the skin substitute is lyophilized, wherein the coloring agent is added to the skin substitute before lyophilization or re-lyophilization of the skin substitute.

12. The tissue-regenerating wound treatment according to any or a combination of 1-11 above or 13-14 below, wherein the coloring agent is added to the skin substitute by dyeing the skin substitute with a dye solution containing 0.01 wt % to 0.0001 wt % of the coloring agent in deionized water or in a phosphate-buffered saline solution.

13. The tissue-regenerating wound treatment according to any or a combination of 1-12 above or 14 below, wherein the coloring agent is characterized by having one or more of antibiotic, antiseptic, antimicrobial, antiviral, antifungal, antiparasitics, anti-inflammatory, or antioxidant properties.

14. The tissue-regenerating wound treatment according to any or a combination of 1-13 above, wherein the coloring agent does not cause a permanent coloring of the wound upon healing.

15. A wound treatment method comprising: providing the tissue-regenerating wound treatment of any or a combination of 1-14 above; applying the tissue-regenerating wound treatment to a wound bed; and determining whether the skin substitute has been degraded by protease attack within the wound by determining a change in color of the coloring agent.

16. A method of producing a tissue-regenerating wound treatment, the method comprising: providing a skin substitute; and adding a coloring agent to the skin substitute, the coloring agent being a biocompatible coloring agent that degrades upon protease attack within a treated wound.

17. The method according to any or a combination of 16 or 18-20 below, wherein the skin substitute is a biological skin substitute, or synthetic substitute, or a hybrid of biological and synthetic skin substitutes, and or the skin substitute is an autologous skin graft, a syngeneic skin graft, an allogeneic skin graft, a xenogeneic skin graft, or a synthetic skin graft, and/or the skin substitute includes a scaffold material, and/or the skin substitute includes a scaffold material that includes an extracellular matrix product.

18. The method according to any or a combination of 16-17 above or 19-20 below, wherein the skin substitute is a scaffold material comprising intact decellularized fish skin, wherein the intact decellularized fish skin comprises extracellular matrix material.

19. The method according to any or a combination of 16-18 above or 20 below, wherein the coloring agent includes methylene blue (MB), or gentian violet (GV), or a combination of methylene blue (MB) and gentian violet (GV).

20. The method according to any or a combination of 16-19 above, wherein the coloring agent is added to the skin substitute by dyeing the skin substitute with a dye solution containing 0.01 wt % to 0.0001 wt % of the coloring agent in deionized water or in a phosphate-buffered saline solution.

To assist in understanding the scope and content of the foregoing written description and appended claims, a select few terms are defined directly below.

As used herein, the term “base material” may include any material known in the art that may act as a vehicle for therapeutics and which may additionally, or alternatively, enable and/or passively regulate moisture at and/or surrounding a wound.

The term “biocompatible polymer” refers to a polymer material which is not harmful to a human body. A biocompatible polymer includes any synthetic or natural polymer material which does not release substances harmful to a human body and which does not cause side effects such as skin stimulation—even when coming in direct contact with and a wound site—or any other negative influence on the human body.

The degrees of “Echelon,” as used herein, refer to locations and/or types of medical attention provided to military personnel. Echelon I refers to self-aid and buddy-aid treatments as well as combat medic treatments administered in the battlefield or at locations remote from Echelon II personnel/facilities. Echelon II refers to advanced trauma care by physicians, physician's assistants, or other qualified medical personnel, and Echelon II care is often administered at a field hospital. Echelon III refers to care provided at the corps level and typically includes reconstructive and definitive surgery to save life, limb, and eyesight; this care may be provided at a field hospital with the necessary equipment. Echelon IV refers to complex surgery and prolonged convalescence (e.g., greater than two weeks) and is generally provided at regional, permanent hospitals. Echelon V refers to injuries and/or procedures that require extensive rehabilitation and convalescent care; Echelon V treatments are administered at continental US permanent hospitals. Although the foregoing Echelon system is particularly relevant to military personnel and treatment scenarios, the Echelon system may also be analogized, as appropriate, to treatment locations and/or types of treatment scenarios in a civilian and/or local law enforcement scenario.

The term “wound” as used herein is intended to encompass tissue injuries generally. Thus, the term “wound” includes those injuries that cause, for example, cutting, tearing, and/or breaking of the skin such as lacerations, abrasions, incisions, punctures, avulsions, or other such injuries. Wounds may be described by any of the size, shape, or magnitude of the wound. For example, a paper cut is exemplary of a small, straight incision of relatively little magnitude, whereas a concussive blast resulting in a major laceration covering one or multiple body parts is exemplary of a relatively larger wound of greater magnitude. Each of the foregoing examples, however, fall within the scope of the term “wound,” as used herein.

The term “wound” additionally includes damage to underlying tissue, such as that caused by traumatic injury. As such, the term “wound” is intended to include a combination of multiple different wounds. For example, a traumatic amputation caused by an explosive blast may generally be referred to as a wound even though it is a compilation of a host of different lacerations, abrasions, avulsions, and punctures. Additionally, any underlying tissue damage resulting from the aforementioned explosive blast may further be encompassed within the understanding of this reference to a wound. The term “wound” is also intended to encompass tissue injuries caused by burns (e.g., thermal and/or chemical burns). Further, the term “wound” is also intended to encompass injuries resulting from, for example, diabetic foot ulcers, venous leg ulcers, surgical operations, pressure ulcers, and other causes.

A “traumatic wound,” as used herein refers to any wound resulting from physical injury that damages both the skin and underlying tissue. A gunshot wound is one non-limiting example of a traumatic wound, as it causes a puncture (i.e., a break) in the skin and ruptures or otherwise damages underlying tissue. As another non-limiting example, a concussive or explosive blast generally results in traumatic wound(s). Many, but not all, of the wounds received during wartime may be described as traumatic wounds due to the nature of war and war-related injuries. A “traumatic wound” can include hemorrhaging wounds, wounds with exposed bone and/or tendons, severe burns, deep tissue wounds (e.g., asymmetrical deep-tissue wounds), and/or large surface area wounds.

Omega3 Wound has been cleared by the Food and Drug Administration (FDA) to use for wound management including chronic wounds, burn wounds, and for soft tissue repair. Unlike other animal-derived products, the fish skin carries no risk of disease transmission to human, thus requires gentle processing where structure and bioactive composition are preserved. Omega3 Wound has demonstrated advantages over porcine small intestinal derived scaffold in faster wound closure and rapid healing time8. The fish skin graft has been used in a large number of chronic and acute wounds in different etiologies and shown robust safety and efficacy. Given the complex and hostile environment of warfare, a holistic approach, in which advanced wound care technologies combined with infection prevention practices, should be taken. It is important that the new technology considers and addresses the needs of soldiers and medical personnel.

Various alterations and/or modifications of the inventive features illustrated herein, and additional applications of the principles illustrated herein, which would occur to one skilled in the relevant art and having possession of this disclosure, can be made to the illustrated embodiments without departing from the spirit and scope of the invention as defined by the claims, and are to be considered within the scope of this disclosure. Thus, while various aspects and embodiments have been disclosed herein, other aspects and embodiments are contemplated. While a number of methods and components similar or equivalent to those described herein can be used to practice embodiments of the present disclosure, only certain components and methods are described herein.

It will also be appreciated that systems, devices, products, kits, methods, and/or processes, according to certain embodiments of the present disclosure may include, incorporate, or otherwise comprise properties, features (e.g., components, members, elements, parts, and/or portions) described in other embodiments disclosed and/or described herein. Accordingly, the various features of certain embodiments can be compatible with, combined with, included in, and/or incorporated into other embodiments of the present disclosure. Thus, disclosure of certain features relative to a specific embodiment of the present disclosure should not be construed as limiting application or inclusion of said features to the specific embodiment. Rather, it will be appreciated that other embodiments can also include said features, members, elements, parts, and/or portions without necessarily departing from the scope of the present disclosure.

Moreover, unless a feature is described as requiring another feature in combination therewith, any feature herein may be combined with any other feature of a same or different embodiment disclosed herein. Furthermore, various well-known aspects of illustrative systems, methods, apparatus, and the like are not described herein in particular detail in order to avoid obscuring aspects of the example embodiments. Such aspects are, however, also contemplated herein.

It is to be understood that not necessarily all objects or advantages may be achieved under an embodiment of the disclosure. Those skilled in the art will recognize that the exoskeletons and methods for making the same may be embodied or carried out in a manner that achieves or optimizes one advantage or group of advantages as taught herein without achieving other objects or advantages as taught or suggested herein.

The skilled artisan will recognize the interchangeability of some of the various disclosed features. Besides the variations described herein, other known equivalents for each feature can be mixed and matched by one of ordinary skill in this art to construct an exoskeleton and utilize a method for making the same under principles of the present disclosure.

Although this disclosure describes certain exemplary embodiments and examples of a passive lumbar exoskeleton, it will be understood by those skilled in the art that the present disclosure extends beyond the specifically disclosed passive lumbar exoskeleton embodiments to other alternative embodiments and/or uses of the disclosure and obvious modifications and equivalents thereof. It is intended that the present disclosure should not be limited by the disclosed embodiments described above and may be extended to other applications that may employ the features described herein.

1. A tissue-regenerating wound treatment comprising:
a skin substitute; and
a coloring agent added to the skin substitute, the coloring agent being a biocompatible coloring agent that degrades upon protease attack within a treated wound.

2. The tissue-regenerating wound treatment according to claim 1, wherein the skin substitute is a biological skin substitute, or synthetic substitute, or a hybrid of biological and synthetic skin substitutes.

3. The tissue-regenerating wound treatment according to claim 1, wherein the skin substitute is an autologous skin graft, a syngeneic skin graft, an allogeneic skin graft, a xenogeneic skin graft, or a synthetic skin graft.

4. The tissue-regenerating wound treatment according to claim 1, wherein the skin substitute includes a scaffold material.

5. The tissue-regenerating wound treatment according to claim 1, wherein the skin substitute includes a scaffold material that includes an extracellular matrix product.

6. The tissue-regenerating wound treatment according to claim 1, wherein the extracellular matrix product is in the form of particles, or a sheet, or a mesh.

7. The tissue-regenerating wound treatment according to claim 1, wherein the skin substitute is a scaffold material comprising intact decellularized fish skin,
wherein the intact decellularized fish skin comprises extracellular matrix material.

8. The tissue-regenerating wound treatment according to claim 1, wherein the wound treatment is crosslinked, before, after, or at a same time that the coloring agent is added to the skin substitute.

9. The tissue-regenerating wound treatment according to claim 1, wherein the coloring agent includes a thiazine dye, or a triarylmethane dye, or a combination of a thiazine dye and a triarylmethane dye.

10. The tissue-regenerating wound treatment according to claim 1, wherein the coloring agent includes methylene blue (MB), or gentian violet (GV), or a combination of methylene blue (MB) and gentian violet (GV).

11. The tissue-regenerating wound treatment according to claim 1, wherein the skin substitute is lyophilized, wherein the coloring agent is added to the skin substitute before lyophilization or re-lyophilization of the skin substitute.

12. The tissue-regenerating wound treatment according to claim 1, wherein the coloring agent is added to the skin substitute by dyeing the skin substitute with a dye solution containing 0.01 wt % to 0.0001 wt % of the coloring agent in deionized water or in a phosphate-buffered saline solution.

13. The tissue-regenerating wound treatment according to claim 1, wherein the coloring agent is characterized by having one or more of antibiotic, antiseptic, antimicrobial, antiviral, antifungal, antiparasitics, anti-inflammatory, or antioxidant properties.

14. The tissue-regenerating wound treatment according to claim 1, wherein the coloring agent does not cause a permanent coloring of the wound upon healing.

15. A wound treatment method comprising:
providing the tissue-regenerating wound treatment of claim 1;
applying the tissue-regenerating wound treatment to a wound bed; and
determining whether the skin substitute has been degraded by protease attack within the wound by determining a change in color of the coloring agent.

16. A method of producing a tissue-regenerating wound treatment, the method comprising:
providing a skin substitute; and
adding a coloring agent to the skin substitute, the coloring agent being a biocompatible coloring agent that degrades upon protease attack within a treated wound.

17. The method according to claim 16, wherein
the skin substitute is a biological skin substitute, or synthetic substitute, or a hybrid of biological and synthetic skin substitutes, and or
the skin substitute is an autologous skin graft, a syngeneic skin graft, an allogeneic skin graft, a xenogeneic skin graft, or a synthetic skin graft, and/or
the skin substitute includes a scaffold material, and/or
the skin substitute includes a scaffold material that includes an extracellular matrix product.

18. The method according to claim 16, wherein the skin substitute is a scaffold material comprising intact decellularized fish skin,
wherein the intact decellularized fish skin comprises extracellular matrix material.

19. The method according to claim 16, wherein the coloring agent includes methylene blue (MB), or gentian violet (GV), or a combination of methylene blue (MB) and gentian violet (GV).

20. The method according to claim 16, wherein the coloring agent is added to the skin substitute by dyeing the skin substitute with a dye solution containing 0.01 wt % to 0.0001 wt % of the coloring agent in deionized water or in a phosphate-buffered saline solution.
